(19) United States
(12) Patent Application Publication
(10) Pub. No.: US 2012/0308570 A1
(43) Pub. Date: Dec. 6, 2012

(54) METHODS FOR TREATING DISEASES

(76) Inventors: Tatiana Kolesnik, Melbourne (AU); Zhihe Kuang, Melbourne (AU); Rowena Sue Lewis, Melbourne (AU); Sandra Elaine Nicholson, Melbourne (AU); Ray Norton, Melbourne (AU)

(21) Appl. No.: 13/319,090

(22) PCT Filed: May 6, 2010
(86) PCT No.: PCT/AU10/00525

§ 371 Date: Nov. 6, 2011

Related U.S. Application Data
(60) Provisional application No. 61/176,637, filed on May 8, 2009.

(51) Int. Cl. A61K 38/17; C07K 7/08; C07K 16/18; A61K 39/395; A61K 38/08; G01N 24/08; A61P 11/00; A61P 29/00; C12N 5/071; G01N 33/566; G01N 33/573; G01N 21/55; C07K 14/435; A61P 11/06

(52) U.S. Cl. 424/139.1; 514/1.7; 530/324; 530/327; 530/387.9; 530/391.1; 530/350; 514/12.2; 435/375; 436/501; 435/7.4

(57) ABSTRACT

The present invention relates to methods of modulating the level of inducible nitric oxide synthase (iNOS) in a cell which comprises administering to the cell a compound which modulates binding of SPRY domain-containing SOCS box protein (SSB) to iNOS, and/or a compound which modulates SSB activity in the cell. Further provided are methods of treating or preventing disease in a subject by modulating the level of iNOS in a cell, as well as compounds which modulate binding of SSB to iNOS and compounds which modulate SSB activity.

### FIELD OF THE INVENTION

The present invention relates to methods of modulating the level of inducible nitric oxide synthase (iNOS) in a cell. The invention also relates to methods of treating or preventing diseases by modulating the level of iNOS in a cell.

### BACKGROUND OF THE INVENTION

Cellular production of reactive nitrogen intermediates (RNI) is an important aspect of the host defence against invading microorganisms. The nitric oxide synthases (NOS) are central to the production of the highly reactive nitric oxide (NO) and the various species 10 produced by its oxidation or reduction (for example NO₂, NO₂⁻, N₂O₃, N₂O₄) which contribute to the killing of intracellular pathogens. Of the three NOS isoforms, nNOS/NOS1 and eNOS/NOS3 (neuronal and endothelial NOS) are dependent on intracellular calcium levels and in general are constitutively expressed, whilst iNOS (or NOS2) is calcium-independent and rapidly induced in response to inflammation and infection.

The active form of iNOS is a homodimer, and a number of cofactors are required for its full activity and the production of NO and citrulline from L-arginine and oxygen. Cytokines and microbial products induce iNOS transcription in macrophages, neutrophils, hepatocytes and endothelial cells, often acting synergistically. For instance, TNFα and the type I or type II interferons, or LPS in combination with IFNγ, significantly enhance iNOS expression. In addition to their role in the innate immune response iNOS and NO have been implicated in a wide spectrum of human physiological responses and diseases including but not limited to autoimmune reactions, tumor growth, and diabetes. The levels of iNOS and NO need to be carefully regulated, with the need for a rapid physiological response balanced with the toxicity associated with excessive or inappropriate NO production.

In many situations it is beneficial to produce nitric oxide in increased amounts. For example, it may be desirable to increase levels of iNOS in cells to promote prophylactic and/or therapeutic actions in regard to diseases or disorders such as microbial infections and cancer. Cytokine induction of iNOS results in production of nitric oxide (NO), and related reactive oxygen intermediates, which are key components of the host defence against pathogens such as Mycobacterium spp. and Leishmania spp.

While nitric oxide has normal intracellular and extracellular regulatory functions, excessive production of nitric oxide can be detrimental in some instances. For example, stimulation of inducible nitric oxide synthesis in blood vessels by bacterial endotoxin, such as, for example, bacterial lipopolysaccharide (LPS) and cytokines that are elevated in sepsis, results in excessive dilation of blood vessels and sustained hypotension commonly encountered with septic shock. Excessive production of nitric oxide is also implicated in diseases such as those involving excessive inflammation, such as immune-mediated arthritis.

There remains a need for methods of modulating the level of iNOS to regulate the production of NO in a cell.

### SUMMARY OF THE INVENTION

The present inventors have identified that SPRY domain-containing SOCS box proteins (SSB) bind to inducible nitric oxide synthetase (iNOS) and act as negative regulators of iNOS.

Accordingly, in one aspect the present invention provides a method of modulating the level of inducible nitric oxide synthetase (iNOS) in a cell, the method comprising administering to the cell a compound which modulates binding of SPRY domain-containing SOCS box protein (SSB) to iNOS, and/or a compound which modulates the level of SSB activity in the cell.

In one embodiment, the method comprises administering to the cell a compound which inhibits binding of SSB to iNOS and/or a compound which reduces the level of SSB activity in the cell, whereby the level of iNOS in the cell is increased.

In another aspect, there is provided a method of treating or preventing a disease in a subject, the method comprising administering a compound which inhibits binding of SSB to iNOS in a cell of the subject and/or a compound which reduces the level of SSB activity in the cell.

In the methods for treating or preventing a disease in a subject, the disease may be one in which it is desirable to have increased levels of nitric oxide (NO). Examples include, but are not limited to, tuberculosis, pneumonia; malaria, listeriosis, amebiasis; candidiasis, trichomoniasis, mycoplasmosis, paracoccidioidomycosis, leishmaniasis, bovine tuberculosis, Johne's disease, porcine enzootic pneumonia, or cancer.

In one embodiment, the disease is caused by infection with Mycobacterium, Salmonella, Toxoplasmasa gondii, Helicobacter pylori, Chlamydia, Chlamydophila, for example Chlamydophila pneumoniae, Staphylococcus; for example Staphylococcus aureus, Escerichia coli, Klebsiella, Pseudomonas, Streptococcus, Burkholderia, for example Burkholderia mallei, Leishmania, Plasmodium or Listeria.

When the disease is caused by infection with Mycobacterium the infection may be, for example, infection with Mycobacterium tuberculosis, Mycobacterium leprae, Mycobacterium lepromatosis, Mycobacterium bovis, Mycobacterium avium, M. avium sub. paratuberculosis or Mycobacterium ulcerans. Where the disease is caused by infection with Mycobacterium, particularly Mycobacterium tuberculosis, the subject is preferably human. Alternatively, in one example, the Mycobacterium is Mycobacterium bovis and the subject is bovine.

When the disease is caused by infection with Plasmodium, the infection may, by way of non-limiting example, be infection with Plasmodium falciparum, Plasmodium vivax, Plasmodium ovale, Plasmodium malariae, or Plasmodium knowlesi.

When the disease is caused by infection with Leishmania, the infection may be, for example, infection with Leishmania major, Leishmania mexicana, Leishmania tropica, Leishmania aethiopica, Leishmania braziliensis, Leishmania donovani, or Leishmania infantum. Where the disease is Leishmaniasis caused by Leishmania infantum, the subject is preferably canine.

In an embodiment, the compound binds to SSB and inhibits the binding of SSB to iNOS.

In one embodiment, the compound is a peptide comprising:

i) an amino acid sequence as provided in any one of SEQ ID NOs:1 to 22,

ii) an amino acid sequence which is at least 80% identical to any one of SEQ ID NOS:1 to 22, and/or

iii) a biologically active fragment of i) or ii).

The peptide may be any length so long as it inhibits the binding of SSB to iNOS and may include the entire sequence of any one of SEQ ID NOs:1 to 22. Alternatively, the peptide may comprise 50 or less, 40 or less, 30 or less, or preferably 20 or less residues.

In one embodiment, the peptide in the methods of the invention consists of a sequence of residues at last 80% identical to any one of SEQ ID NOs:1 to 22. Preferably, the peptide may be at least 85%, 90%, 95% or 99% identical to any one of SEQ ID NOs:1 to 22.

In another embodiment, the compound is a mimetic of the peptide as described herein.

In yet another embodiment, the compound which modulates binding of SSB to iNOS, and/or the compound which modulates the level of SSB activity in the cell is an antibody that binds SSB.

Preferably, the antibody binds to amino acid residues within:

i) an amino acid sequence as provided in any one of SEQ ID NOs:64 to 82, and/or

ii) an amino acid sequence which is at least 80% identical to any one of SEQ ID

NOs:64 to 82.

In one embodiment, the antibody binds to one or more of residues E55, N56, R68, P70, A72, R100, G101, T102, H103, Y120, L123, L124, L125, S126, N127, S128, V206, W207 or G208 of SEQ ID NO:64, or to an epitope which comprises one or more of said residues.

In yet another embodiment of the methods of the invention, the compound is functionally inactive iNOS, or an isolated polynucleotide encoding the functionally inactive iNOS.

In another embodiment, the compound binds to iNOS and inhibits the binding of iNOS to SSB.

A polypeptide comprising modified SSB that includes the SPRY domain, but which does not have SSB activity, would compete with native SSB for binding to iNOS. Thus, in one embodiment, the compound is an isolated polypeptide comprising the SPRY domain of SSB, or an isolated polynucleotide encoding the polypeptide, wherein the polypeptide does not have SSB activity. Preferably, the polypeptide comprises an amino acid sequence at least 80% identical to any one of SEQ ID NOs:64 to 82.

In yet another embodiment, the compound is an antibody which binds iNOS and inhibits binding of iNOS to SSB in a cell.

Preferably, the antibody binds to amino acid residues within:

i) an amino acid sequence as provided in any one of SEQ ID NOs:1 to 22, and/or

ii) an amino acid sequence which is at least 80% identical to any one of SEQ ID NOs:1 to 22.

In yet another embodiment of the invention, the compound which modulates the level of SSB activity in the cell is an isolated polynucleotide which reduces the level of SSB activity in the cell and/or construct encoding said polynucleotide. The polynucleotide may be, for example, an antisense polynucleotide, a sense polynucleotide, a catalytic polynucleotide, a microRNA, and a double-stranded RNA. By way of example, the double-stranded RNA may be a siRNA or shRNA.

In one particular embodiment, the polynucleotide comprises a sequence of nucleotides at least 90% identical to SEQ ID NO:84.

In some instances it may be desirable to reduce the level of iNOS in a cell, for example, in a subject suffering from sepsis-induced lung injury, asthma, shock, excessive inflammation and/or excessive cytokine production. Thus, in one embodiment, the method comprises administering to the cell a compound which increases SSB activity in the cell, whereby the level of iNOS in the cell is reduced.

In a further aspect, the present invention provides a method of treating or preventing a disease in a subject, the method comprising administering to the cell a compound which increases SSB activity in the cell, whereby the level of iNOS in the cell is reduced.

In one embodiment, the disease that is treated or prevented is sepsis-induced lung injury, asthma, shock, for example, septic shock, post-operative hypotension, hypovolaemic shock, neurogenic shock, cardiogenic shock, distributive shock, combined shock; or is caused by excessive inflammation, for example rheumatoid arthritis, systemic lupus erythematosus, other organ specific inflammation, reperfusion injury, for example repurfusion injury following revascularisation procedures for an ischaemic limb or reperfusion injury following stroke; and/or excessive cytokine production including toxic shock syndrome. The cytokine that is produced in excess may be, for example but not limited to, TNFα, IFNγ, or type I interferons (IFNα/β).

In another embodiment, the compound is an isolated polypeptide comprising the SPRY domain and SOCS box of SSB, or a polynucleotide encoding the polypeptide, wherein the polypeptide has SSB activity. In one particular embodiment, the polypeptide is SSB.

In the methods of the invention, the SSB is preferably SSB-1, 2 or 4, more preferably SSB-2 or 4 and most preferably SSB-2.

The cell may be any cell that produces SSB and iNOS. In one embodiment, the cell is a T-cell, dendritic cell, macrophage or a neutrophil. In a preferred embodiment, the cell is a macrophage.

In another aspect, the present invention provides an isolated peptide or mimetic thereof, wherein the peptide consists of:

i) an amino acid sequence as provided in any one of SEQ ID NOs:1 to 22

ii) an amino acid sequence which is at least 80% identical to any one of SEQ ID NOs:1 to 22, and/or

iii) a biologically active fragment of i) or ii).

Preferably, the peptide may be at least 85%, 90%, 95% or 99% identical to any one of SEQ ID NOs:1 to 22

In one embodiment, the isolated peptide is 20 or less residues in length.

In another embodiment, the isolated peptide or mimetic thereof is a retro-inverso peptide. In one example, the isolated peptide or mimetic is a retro-inverso peptide of any one of SEQ ID NOS:1-22.

In a further aspect, the present invention provides an isolated antibody which binds to SSB and inhibits binding of SSB to iNOS in a cell

Preferably, the antibody binds to amino acid residues within:

i) an amino acid sequence as provided in any one of SEQ ID NOs:64 to 82, and/or

ii) an amino acid sequence which is at least 80% identical to any one of SEQ ID NOs:64 to 82.

In one embodiment, the antibody binds to one or more of residues E55, N56, R68, P70, A72, R100, G101, T102, H103, Y120, L123, L124, L125, S126, N127, S128, V206, W207 or G208 of SEQ ID NO:64, or to an epitope which comprises one or more of said residues.

The present invention further provides an isolated antibody which binds iNOS and inhibits binding of iNOS to SSB in a cell.

Preferably, the antibody binds to amino acid residues within:

i) an amino acid sequence as provided in any one of SEQ ID NOs:1 to 22, or

ii) an amino acid sequence which is at least 80% identical to any one of SEQ ID NOs:1 to 22.

In another embodiment, the compound which modulates binding of SSB to iNOS and/or which modulates SSB activity in the cell is fused and/or conjugated to a macrophage or T-cell targeting agent or a cell penetrating agent. In one particular embodiment, the peptide or mimetic thereof of the invention or the antibody of the invention is fused and/or conjugated to a macrophage or T-cell targeting agent or a cell penetrating agent.

The present invention further provides use of a compound which inhibits binding of SSB to iNOS in a cell and/or a compound which reduces the level of SSB activity in a cell for the manufacture of a medicament for treating or preventing a disease in a subject.

The present invention further provides use of a compound which increases SSB activity in a cell for the manufacture of a medicament for treating or preventing a disease in a subject.

In another aspect, the invention provides a pharmaceutical composition comprising the peptide or mimetic thereof of the invention and/or the antibody of the invention.

In yet another aspect, the invention provides the peptide or mimetic thereof of the invention, the antibody of the invention, and/or the pharmaceutical composition of the invention for use as a medicament.

In a further aspect, the present invention provides a method for identifying an inhibitor of the binding of SSB to iNOS, the method comprises the steps of:

i) contacting SSB, or an iNOS binding fragment thereof, or iNOS, or a SSB binding fragment thereof, with one or more candidate compounds,

ii) identifying a candidate compound which binds to SSB or iNOS, and

iii) determining whether the candidate compound inhibits the binding of SSB to iNOS.

In one embodiment, the candidate compound which binds to SSB or iNOS is identified by surface plasmon resonance or high-resolution NMR.

In another embodiment, step iii) comprises:

a) incubating iNOS, or a SSB binding fragment thereof, with SSB, or an iNOS binding fragment thereof, with the candidate compound under conditions sufficient for SSB to bind to iNOS to form a complex, and

b) determining if the candidate compound inhibits the formation of the complex.

Preferably, the candidate compound is a peptide or mimetic thereof, or an antibody.

As the skilled person will appreciate, the candidate compound may bind to SSB, or to iNOS.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures. Throughout the text SPSB is used interchangeably with SSB. spsb refers to the SSB (SPSB) gene.

### BRIEF DESCRIPTION OF THE ACCOMPANYING DRAWINGS

Some figures contain coloured representations or entities. Coloured versions of the figures are available from the Patentee upon request or from an appropriate Patent Office. A fee may be imposed if obtained from a Patent Office.

FIG. 1. Alignment of SSB SPRY domain amino acid sequences from several species with Drosophila GUSTAVUS (SEQ ID NO:71) sequence.

FIG. 2. Amino acid sequence alignments of the iNOS (NOS2) proteins. The EKDINNNVXK (SEQ ID NO:35) motif is conserved in iNOS but is not present in either eNOS or iNOS(NOS1, data not shown). The sequence of the mouse iNOS N-terminal peptide (SEQ ID NO:3) used in ITC and NMR experiments is indicated.

FIG. 3. Typical ITC raw data and titration curves for SSB-2 and iNOS peptide interactions. (I-XV) iNOS peptides are as listed in Table 2. Titration curves were fitted using the “One Set of Sites” model in MicroCal Origin.

FIG. 4. Interaction between SSB-2ΔSB and iNOS N-terminal peptide analysed by NMR spectroscopy. (A) Overlay of the ¹H-¹⁵N HSQC spectra of 0.1 mM ¹⁵N-labelled SSB-2ΔSB in the absence and presence of unlabelled iNOS peptide at SSB-2ΔSB:iNOS peptide molar ratios of 1:1.5. Samples were in 95% H₂O/5% ²H₂O containing 10 mM sodium phosphate, 50 mM sodium chloride, 2 mM EDTA, 2 mM DTT and 0.02% (w/v) sodium azide at pH 7.0. Spectra were recorded at 500 MHz and 22° C. (B) Ribbon model of SSB-2ΔSB (PDB ID code 3EK9) showing residues whose ¹H-¹⁵N cross-peaks had relatively large chemical shift perturbations upon iNOS peptide binding.

FIG. 5. SSB-2 interacts with endogenous full-length iNOS protein. (A) SSB-2 interacts with full-length iNOS and this requires tyrosine 120 in the SPRY domain peptide-binding surface. Bone marrow-derived macrophages (BMDM) from C57BL/6 mice were incubated with 20 ng/ml IFNγ and 1 μg/ml LPS for 16 h, lysed and incubated with NHS-sepharose beads coupled with recombinant SSB-2 or SSB-2-Y120A proteins, or with uncoupled NHS-sepharose beads (CON), for 3 h at 4° C. Associated proteins were then separated by SDS-PAGE and transferred to PVDF membrane. iNOS was detected by Western blot with specific anti-iNOS antibodies (upper panel). Equivalent amounts of SSB-2 and SSB-2-Y120A were confirmed by reprobe with anti-SSB-2 antibodies (lower panel). (B) Interaction between endogenous iNOS and SSB-2 proteins. Bone marrow-derived macrophages from SSB-2-deficient mice (Ssb-2^{-/-}) or wild-type littermate controls (Ssb-2^{+/+}) were incubated with (+) or without (−) 20 ng/ml IFNγ and 20 ng/ml LPS for 16 h, lysed and endogenous SSB-2 proteins immunoprecipitated using rabbit anti-SSB-2 antibody coupled to NHS-Sepharose. Immunoprecipitates were separated by SDS-PAGE and associated iNOS protein detected by Western blot with specific antibodies (upper panel). Membranes were stripped and reprobed using biotinylated anti-SSB-2 protein (middle panel). iNOS induction was confirmed by Western blot of protein lysates using anti-iNOS antibodies (lower panel).

FIG. 6. Interaction between iNOS and SSB-1, -2, and -4 and SSB-2 residues affecting 10 iNOS binding. (A) iNOS interacts preferentially with SSB-2 and SSB-4. 293T cells were transiently transfected with vector alone or cDNA encoding either Flag-tagged SSB-1, SSB-2, SSB-3 or SSB-4. Cells were lysed, and mixed with BMDM lysates from cells induced to express iNOS. Flag-tagged proteins were immunoprecipitated using anti-Flag antibodies (M2-beads) and separated by SDSPAGE. Co-immunoprecipitation of iNOS was detected by Western blot with anti-iNOS antibodies (upper panel). Membranes were stripped and reprobed with rat anti-Flag antibodies (middle panel). Comparative expression of Flag-tagged proteins in 293T lysates is shown by Western blot (lower panel). (B) SSB-2 residues affecting iNOS binding. Residues in the SSB-2 SPRY domain were mutated to Ala or Phe (for Y120) based on SSB-2 structure and sequence conservation. 293T cells 20 were transiently transfected with cDNA encoding Flag-tagged wild-type or mutant SSB-2. Cells were lysed, and mixed with BMDM lysates from cells induced to express iNOS. Flag-tagged proteins were immunoprecipitated using anti-Flag antibodies and separated by SDS-PAGE. Coimmunoprecipitation of iNOS was detected by Western blot with anti-iNOS antibodies.

FIG. 7. Expression of SSB-1 mRNA is rapidly and transiently induced in response to LPS and IFNγ. BMDM were incubated in medium containing M-CSF (L-cell conditioned medium) and 1 μg/ml LPS/10 ng/ml IFNγ (A) or 20 ng/ml LPS/IFNγ (B & C) for the times indicated. In (B) cells were washed after 24 h incubation and replenished with fresh medium containing L-cell conditioned medium. Total RNA was extracted and SSB or iNOS mRNA levels analysed by Q-PCR (normalized against GAPDH mRNA levels). All points represent means and standard deviations from macrophage cultures derived from three individual mice.

FIG. 8. iNOS clearance is reduced post-stimulus in SSB-2 deficient macrophages. (A). BMDM from SSB-2-deficient mice (Ssb-2^{-/-}) or littermate controls (Ssb-2^{+/+}) were incubated with IFNγ and LPS (20 ng/ml) for the times indicated. (B) BMDM from either Ssb-2^{+/+} or Ssb-2^{-/-} mice were incubated with or without (−) IFNγ and LPS (20 ng/ml) for 16 h, washed, replenished with fresh medium and lysed at the indicated times post-wash. Lysates were then separated by SDS-PAGE and analysed by Western blot using anti-iNOS antibodies (upper. panels). Equivalent protein loading was confirmed by stripping and reprobing membranes with anti-tubulin antibodies (lower panels).

FIG. 9. iNOS levels are reduced in macrophages derived from SSB-2 transgenic mice and this requires the SSB-2 SOCS box. BMDM from littermate controls (Ssb-2^{+/+}) and SSB-2-trangenic mice (Ssb-2^{T/+}) (A) or from Ssb-2^{+/+} and SSB-2-transgenic mice lacking the SOCS box (Ssb-2ΔSB^{T/+}) (B) were incubated with or without (−) 20 ng/ml LPS/IFNγ for 16 h, washed, replenished with fresh medium and lysed at the indicated times post-wash. Proteins were then separated by SDS-PAGE and analysed by Western blot using anti-iNOS antibodies (upper panels). Equivalent protein loading was confirmed by stripping and reprobing membranes with anti-tubulin antibodies (lower panels). (C) Expression of Flag-tagged SSB-2 and SSB-2ΔSB transgenes in LPS/IFN-γ-treated BMDM from Ssb-2^{T/+} and Ssb-2ΔSB^{T/+} mice respectively, was confirmed by anti-Flag immunoprecipitation and Western blot (upper panel). Membranes were stripped and reprobed for iNOS association (upper middle panel). iNOS expression was confirmed by Western blot of cell lysates (lower middle panel) and equivalent protein levels by Western blot with anti-tubulin antibodies (lower panel).

FIG. 10. iNOS levels are reduced in macrophages derived from SSB-1 transgenic mice and this requires the SSB-1 SOCS box. BMDM from wild-type littermates (Ssb-1^{+/+}) and SSB-1-transgenic mice (Ssb-1^{T/+}) (A) or from Ssb-1^{T/+} and SSB-1-transgenic mice lacking the SOCS box (Ssb-1ΔSB^{T/+}) (B) were incubated with or without (−) 20 ng/ml LPS/IFNγ for 16 h, washed, replenished with fresh medium and lysed at the indicated times post-wash. Proteins were then separated by SDS-PAGE and analysed by Western blot using anti-iNOS antibodies (upper panels). Equivalent protein loading was confirmed by stripping and reprobing membranes with anti-tubulin antibodies (lower panels). (C) Expression of Flag-tagged SSB-1 and SSB-1ΔSB transgenes in LPS/IFN-γ-treated BMDM from Ssb-1^{T/+} and Ssb-1ΔSB^{T/+} mice respectively, was confirmed by anti-Flag immunoprecipitation and Western blot (upper panel). Membranes were stripped and reprobed for iNOS association (upper middle panel). iNOS expression was confirmed by Western blot of cell lysates (lower middle panel) and equivalent protein levels by Western blot with anti-tubulin antibodies (lower panel).

FIG. 11. SSB-1 and SSB-2 regulation of iNOS expression is dependent on the proteasome. BMDM from (A) littermate controls (Ssb-1^{+/+}) and SSB-1-transgenic mice (Ssb-1^{T/+}) or (B) Ssb-1^{+/+} and SSB-2-transgenic mice (Ssb-2^{T/+}) were incubated with IFNγ and LPS (20 ng/ml) for 16 h, washed, replenished with fresh medium with (+) or without (−) the proteasomal inhibitor MG-132 (10 μM) and lysed at the indicated times post-wash. Proteins were separated by SDS-PAGE and analysed by Western blot using anti-iNOS antibodies. Equivalent protein loading was confirmed by stripping and reprobing the membrane with anti-tubulin antibodies.

FIG. 12. Nitric oxide production in bone marrow-derived SSB-2-deficient and SSB-2-overexpressing macrophages. BMDM from C57BL/6, SSB-2-deficient (Ssb-2^{-/-}), SSB-2-transgenic (Ssb-2^{T/+}) and SSB-2-transgenic mice lacking the SOCS box (Ssb-2DSB^{T/+}) were cultured for 24 h in medium containing either 2 or 20 ng/ml LPS. Aliquots of culture supernatant were then assayed for nitric oxide by Griess assay. Data are shown as mean±standard deviation. n=3 where each replicate represents cells derived from independent mice. *p<0.05

FIG. 13. iNOS peptide (SEQ ID NO:3) can competitively inhibit the iNOS/SSB-2 interaction and iNOS ubiquitination. (A). 293T cells were transiently transfected with cDNA expressing Flag-tagged SSB-2, lysed and mixed with iNOS-expressing macrophage lysates containing increasing amounts of free iNOS peptide. Anti-Flag immunoprecipitates were then assessed for iNOS interaction by SDS-PAGE and Western blot with anti-iNOS antibodies. (B) An in vitro ubiquitination assay was performed using recombinant E1, E2 and E3 ligase components and macrophage lysates as a source of iNOS. Excess free iNOS peptide was added as indicated. The reaction mixture was then separated by SDS-PAGE 20 and analysed by Western blot with anti-iNOS antibodies (upper panel) or by Coomassie stain (lower panel).

FIG. 14. Increased levels of iNOS result in enhanced nitric oxide production in peritoneal macrophages. (A) Thioglycollate-elicited peritoneal macrophages from SSB-2-deficient mice (Ssb-2^{-/-}) and littermate control mice (Ssb-2^{+/+}), were cultured for 16 h in medium containing 20 ng/ml LPS/IFNγ, washed, replenished with fresh medium and lysed at the indicated times post-wash. Proteins were separated by SDS-PAGE and analysed by Western blot with anti-iNOS antibodies (upper panel). Equivalent protein loading was confirmed by stripping and reprobing membranes with anti-tubulin antibodies (lower panel). (B) Peritoneal macrophages were cultured for 24 h in medium containing either 2 or 20 ng/ml LPS. Aliquots of culture supernatant were then assayed for nitric oxide by Griess assay. Data are shown as mean±standard deviation. n≧3 where each replicate represents cells derived from independent mice. *p<0.05.

FIG. 15. SSB-2-deficient macrophages show enhanced killing of Leishmania major parasites. (A) BMDM from Spsb2^{+/+}, Spsb2^{-/-} and Spsb2^{T/+} mice were incubated in the presence of Leishmania major promastigotes, with or without 10 ng/ml IFN-γ for 48 h. Culture supernatants were then assayed for NO production. Data are shown as mean of triplicate cultures±standard deviation and are representative of four separate experiments. (B & C) BMDM from Spsb2^{+/+} and Spsb2^{-/-} mice were infected with Leishmania major promastigotes. The percentage of infected cells was determined at 5 and 48 h post-infection. (C) 5 μM of the iNOS inhibitor, 1400 W was added to cultures 5 h post-infection. Data are shown as mean±standard deviation (n=3, where each replicate represents cells derived from individual mice). *p<0.05, **p<0.005.

FIG. 16. iNOS is induced earlier and to a greater magnitude in BMDM with reduced expression of SPSB1. BMDM from C57BL/6 mice were infected with either non-sense control shRNA or Spsb1 shRNA and incubated with or without 10 ng/ml LPS for 4 h, lysed and analysed. for expression of Spsb1 via Q-PCR (A). Alternatively, BMDM were incubated with or without 100 ng/ml LPS (B) or 25 μg/ml PolyIC (C) for the times indicated, or incubated with or without 20 ng/ml LPS (D) or 25 μg/ml PolyIC (E) overnight. In (D) and (E) cells were washed, replenished with fresh medium and lysed at the indicated times post-wash. Lysates were then separated by SDS-PAGE and analysed by Western blot using anti-iNOS antibodies (upper panels). Equivalent protein loading was confirmed by stripping and reprobing membranes with anti-ERK antibodies (lower panels).

FIG. 17. Nitric oxide production is increased in Spsb1 shRNA infected BMDM. BMDM from C57BL/6 mice were infected with either non-sense control shRNA or Spsb1 shRNA and cultured in medium containing either 100 ng/ml LPS or 25 μg/ml PolyIC. Culture supernatants were assayed for nitric oxide by the Griess assay at 24 h (A) or 48 h (B). Data are shown as mean±standard deviation (n=3).

FIG. 18. Expression analysis of Spsb genes in response to TLR agonists and TGFβ. BMDM were generated from C57BL/6 mice and incubated in medium containing M-CSF (L-cell conditioned medium) and either 10 ng/ml LPS (A), 10 μg/ml PolyIC or 10 ng/ml Pam3Cys (B), 1000 U/ml IFNα, 1000 U/ml IFNβ (E) or 10 ng/ml TGFβ (F). BMDM were derived from C57BL/6, TRIF −/− (KO) or MyD88−/− (KO) mice and incubated in medium containing M-CSF (L-cell conditioned medium) and 10 ng/ml LPS(C) or 10 μg/ml PolyIC (D) over an 8 h period. Total RNA was extracted and SPSB mRNA levels analysed by Q-PCR (normalised against GAPDH). All points represent mean and standard deviations from macrophage cultures derived from three individual mice.

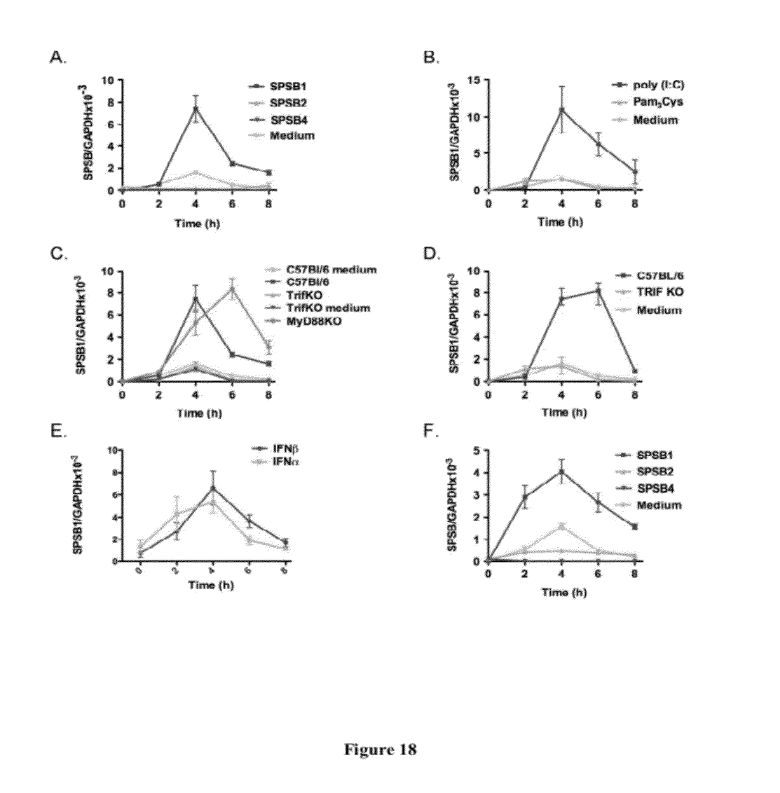

FIG. 19. (A) BMDM from C57BL/6, Spsb2^{-/-}, Spsb2^{T/+} and Spsb2ΔSB^{T/+} mice were pre-incubated with or without 20 ng/ml IFN-γ, washed with PBS, and infected with Listeria monocytogenes in DMEM without antibiotics for 30 min. Cells were then washed and cultured in DMEM containing 10 μg/ml gentamicin for 16 h. Data are shown as mean±standard deviation (n≧3, where each replicate represents cells derived from individual mice). (B) BMDM from Spsb2^{+/+}, Spsb2^{-/-} and Spsb2^{T/+} mice were stimulated overnight with (+) or without (−) IFN-γ, then infected with M. bovis BCG for 2 h, extracellular 40 bacteria removed and supernatants were assayed for NO production 24 and 48 h post infection. Data are shown as mean±standard deviation of quadruplicate cultures from 1 of 2 experiments. *p<0.05.

### KEY TO THE SEQUENCE LISTING

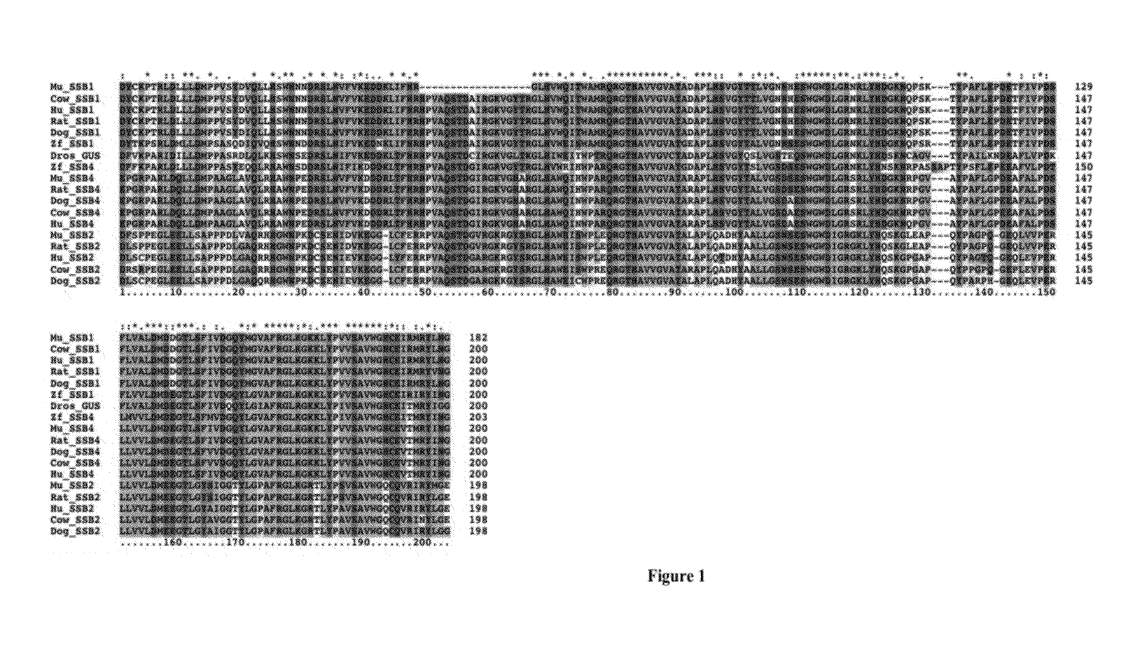

SEQ ID NO:1—amino acid sequence of N-terminal region of human iNOS.
SEQ ID NO:2—human N-terminal iNOS motif.
SEQ ID NOs:3-17—iNOS N-terminal peptides (see Table 2).
SEQ ID NO:18—rat N-terminal iNOS motif.
SEQ ID NO:19—bovine N-terminal iNOS motif.
SEQ ID NO:20—canine N-terminal iNOS motif.
SEQ ID NO:21—guinea pig N-terminal iNOS motif.
SEQ ID NO:22—chicken N-terminal iNOS motif.
SEQ ID NOs:23-34—Oligonucleotide primers.
SEQ ID NO:35—motif sequence.
SEQ ID NO:36—motif sequence.
SEQ ID NO:37—Flag epitope.
SEQ ID NO:38—vector N-terminus residues 6-11.
SEQ ID NO:39—vector C-terminus residues 225-231.
SEQ ID NO:40—human SSB-2 mRNA.
SEQ ID NO:41—human SSB-2.
SEQ ID NO:42—mouse SSB-2 mRNA.
SEQ ID NO:43—mouse SSB-2.
SEQ ID NO:44—canine SSB-2 mRNA.
SEQ ID NO:45—canine SSB-2.
SEQ ID NO:46—human SSB-1 mRNA.
SEQ ID NO:47—human SSB-1.
SEQ ID NO:48—mouse SSSB-1 mRNA.
SEQ ID NO:49—mouse SSB-1.
SEQ ID NO:50—canine SSB-1 mRNA.
SEQ ID NO:51—canine SSB-1.
SEQ ID NO:52—human SSB-4 mRNA.
SEQ ID NO:53—human SSB-4:
SEQ ID NO:54—mouse SSB-4 mRNA.
SEQ ID NO:55—mouse SSB-4.
SEQ ID NO:56—canine SSB-4 mRNA.
SEQ ID NO:57—canine SSB-4.
SEQ ID NO:58—human iNOS.
SEQ ID NO:59—mouse iNOS.
SEQ ID NO:60—canine iNOS.
SEQ ID NO:61—bovine iNOS.
SEQ ID NO:62—chicken iNOS.
SEQ ID NO:63—rat iNOS.
SEQ ID NO:64—SPRY domain of human SSB-2.
SEQ ID NO:65—SPRY domain of mouse SSB-1 (see FIG. 1).
SEQ ID NO:66—SPRY domain of bovine SSB-1 (see FIG. 1).
SEQ ID NO:67—SPRY domain of human SSB-1 (see FIG. 1).
SEQ ID NO:68—SPRY domain of rat SSB-1 (see FIG. 1).
SEQ ID NO:69—SPRY domain of canine SSB-1 (see FIG. 1).
SEQ ID NO:67—SPRY domain of zebra fish SSB-1 (see FIG. 1).

### SEQ ID NO:71—Drosophila GUSTAVUS (see FIG. 1).

SEQ ID NO:72—SPRY domain of zebra fish SSB-4 (see FIG. 1).
SEQ ID NO:73—SPRY domain of mouse SSB-4 (see FIG. 1).
SEQ ID NO:74—SPRY domain of rat SSB-4 (see FIG. 1).
SEQ ID NO:75—SPRY domain of canine SSB-4 (see FIG. 1).
SEQ ID NO:76—SPRY domain of bovine SSB-4 (see FIG. 1).
SEQ ID NO:77—SPRY domain of human SSB-4 (see FIG. 1).
SEQ ID NO:78—SPRY domain of mouse SSB-2 (see FIG. 1).
SEQ ID NO:79—SPRY domain of rat SSB-2 (see FIG. 1).
SEQ ID NO:80—SPRY domain of human SSB-2 (see FIG. 1).
SEQ ID NO:81—SPRY domain of bovine SSB-2 (see FIG. 1).
SEQ ID NO:82-SPRY domain of canine SSB-2 (see FIG. 1).
SEQ ID NO:83—SOCS box of human SSB-2.
SEQ ID NO:84—shRNA targeting Spsb 1.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in protein chemistry, biochemistry, cell culture, molecular genetics, microbiology, immunology and immunohistochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols. in Immunology, John Wiley & Sons (including all updates until present).

“SSB” as used herein refers to a polypeptide belonging to the mammalian SPRY domain-containing SOCS box protein family (SSB-1 to -4; see for example Hilton et al., 1998). The official gene name for this family is Spsb1-4. Throughout the text of this specification the terms SSB and SPSB may be used interchangeably (spsb refers to the gene encoding SSB/SPSB). The SOCS box motif recruits an E3 ubiquitin ligase complex, which polyubiquitinates proteins targeted by interaction with the SPRY protein interaction domain, resulting in their proteasomal degradation. Examples of SSB proteins include the human proteins SSB-1 (SEQ ID NO:47), SSB-2 (SEQ ID NO:41), SSB-3 and SSB-4 (SEQ ID NO:53), as well as orthologous molecules in other animals such as, for example, dog (SEQ ID NOs:45, 51 and 57) and mouse (SEQ ID NOs:43, 49 and 55). The SPRY domain is involved in iNOS binding and in SSB-2 comprises amino acid residues 26-221 (SEQ ID NO:64). The SOCS box is required for recruitment of an E3 ubiquitin ligase complex and in SSB-2 comprises amino acid residues 222-263 (SEQ ID NO:83). This complex polyubiquitinates iNOS resulting in its degradation.

“SSB activity” as used herein refers to the ability of a polypeptide to bind to iNOS and associate with the E3 ubiquitin ligase complex.

As used herein “iNOS” refers to inducible nitric oxide synthase (NCBI Accession No. P35228; also referred to as NOS2) and includes human iNOS (SEQ ID NO:58), as well as orthologous molecules in other organisms, for example murine iNOS (SEQ ID NO:59), canine iNOS (SEQ ID NO:60), bovine iNOS (SEQ ID NO:60), avian iNOS (SEQ ID NO:61) iNOS and rat iNOS (SEQ ID NO:63).

The terms “protein”, “polypeptide” and “peptide” are generally used interchangeably. However, the term “peptide” is typically used to refer to chains of amino acids which are not large, for instance 100 or less residues in length.

As used herein a “biologically active fragment” is a portion of a polypeptide or peptide as described herein which maintains a defined activity of the full-length polypeptide. Biologically active fragments can be any size as long as they maintain the defined activity. With regard to the peptides described herein, a preferred biological activity is binding to SSB or iNOS.

As used herein, the term “epitope” refers to a region of a peptide or polypeptide as described herein which is bound by an antibody.

As used herein, the term “subject” relates to an animal. More preferably, the subject is a mammal such as a human, dog, cat, horse, cow, or sheep. Alternatively, the subject may be avian, for example, poultry such as a chicken, turkey or duck. Most preferably, the subject is a human.

By “inhibits” or “inhibiting” binding is meant a decrease or reduction in binding of SSB to iNOS in the presence of a compound, for example a compound of the invention, when compared to binding of SSB to iNOS in the absence of the compound, such as in a control sample. The degree of decrease or inhibition of binding will vary with the nature and quantity of the compound present, but will be evident e.g., as a detectable decrease in binding of SSB to iNOS; desirably a degree of decrease greater than 10%, 33%, 50%, 75%, 90%, 95% or 99% as compared to binding of SSB to iNOS in the absence of the compound.

By “reduces” or “reducing” the level or activity of SSB or iNOS in a cell is meant a decrease in the amount or activity of SSB or iNOS in a cell in the presence of a compound, for example a compound of the invention, when compared to the amount or activity of SSB or iNOS in the cell in the absence of the compound, such as in a control sample. The degree of decrease in the amount or activity of SSB or iNOS will vary with the nature and quantity of the compound present, but will be evident e.g., as a detectable decrease in the amount or activity of SSB or iNOS; desirably a degree of decrease greater than 10%, 33%, 50%, 75%, 90%, 95% or 99% as compared to the amount or activity of SSB or iNOS in the absence of the compound.

“Administering” as used herein is to be construed broadly and includes administering a compound as described herein to a subject as well as providing a compound as described herein to a cell.

As used herein, the terms “treating”, “treat” or “treatment” include administering a therapeutically effective amount of an compound as described herein sufficient to reduce or delay the onset or progression of specified disease, or to reduce or eliminate at least one symptom of the disease.

As used herein, the terms “preventing”, “prevent” or “prevention” include administering a therapeutically effective amount of a compound useful for the invention sufficient to stop or hinder the development of at least one symptom of the specified condition.

As used herein, the terms “conjugate”, “conjugated” or variations thereof are used broadly to refer to any form to covalent or non-covalent association between a compound useful for the invention and another agent.

As used herein, the term “cell targeting agent” refers to any agent capable of targeting a compound as described herein to a cell. The term “macrophage targeting agent” refers to any agent capable of targeting a compound as described herein to a macrophage in vivo, the term “T-cell targeting agent” refers to any agent capable of targeting a compound as described herein to a T-cell in vivo, the term “dendritic cell targeting agent” refers to any agent capable of targeting a compound as described herein to a dendritic cell in vivo, and the term “neutrophil targeting agent” refers to any agent capable of targeting a compound as described herein to a neutrophil in vivo. Cell targeting agents include for example, phospholipids, liposomes, microspheres, nanoparticles, mannose, mannose-6-phosphate, lactose, galactose, N-acetyl-galactosamine, glycoproteins, lectins, melanotropin, thyrotropin, or antibodies to macrophage, T-cell, dendritic cell and/or neutrophil surface molecules.

As used herein, the term “cell penetrating agent” includes compounds or functional groups which mediate transfer of a substance from an extracellular space to an intracellular compartment of a cell. For example, a cell penetrating moiety may be a hydrophobic moiety and the hydrophobic moiety may be, e.g., a mixed sequence peptide or a homopolymer peptide such as polyleucine or polyarginine at least about 11 amino acids long. Examples of cell penetrating peptides include Tat peptides, Penetratin, short amphipathic peptides such as those from the Pep- and MPG-families, oligoarginine and oligolysine Alternatively, the cell-penetrating agent may be a lipid such as a straight chain fatty acid.

Compounds for Modulating Binding of SSB to iNOS

Modified SSB or iNOS

In one embodiment of the invention, the compound which modulates binding of SSB to iNOS is a polypeptide comprising modified SSB lacking SSB activity that binds to iNOS and inhibits SSB binding to iNOS. By way of example, the polypeptide may comprise the SPRY domain of SSB required for iNOS binding, but does not comprise the SOCS box that is required for association with the E3 ligase complex and subsequent degradation of iNOS. The polypeptide will compete with native SSB for binding to iNOS, resulting in an increased level of iNOS in the cell. Preferably, the polypeptide comprises an amino acid sequence at least 80% identical to any one of SEQ ID NOs:64 to 82. An alignment of the SSB SPRY domain from several species (SEQ ID NOs:64 to 82) is provided in FIG. 1.

With regard to a defined polypeptide or peptide, it will be appreciated that % identity figures higher than those provided above will encompass preferred embodiments. Thus, where applicable, in light of the minimum % identity figures, it is preferred that the polypeptide or peptide comprises an amino acid sequence which is at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

The % identity of a polypeptide can be determined by GAP (Needleman and Wunsch, 1970) analysis (GCG program) with a gap creation penalty=5, and a gap. extension penalty=0.3. Preferably, the query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP. analysis aligns the two sequences over a region of at least 100 amino acids. Preferably, the two sequences are aligned over their entire length.

In another embodiment, the compound is functionally inactive iNOS that binds to SSB and inhibits SSB binding to iNOS. By “functionally inactive iNOS” is meant iNOS which is modified compared to native iNOS and which is not capable of producing nitric oxide in vivo. Thus, the functionally inactive iNOS competes with native iNOS for binding to SSB in a cell, resulting in an increase in iNOS in the cell.

The person skilled in the art will appreciate that the functionally inactive iNOS or fragment of SSB described herein may be administered to a cell in any suitable form, including as a polynucleotide encoding the functionally inactive iNOS or fragment of SSB.

Peptides and Mimetics Thereof

In another embodiment, the compound which inhibits binding of SSB to iNOS is a peptide or a mimetic thereof derived from the amino acid sequence of iNOS or SSB.

In one embodiment, candidate compounds are peptides of from about 5 to about 30 amino acids, or from about 5 to about 20 amino acids, or from about 7 to about 15 amino acids. In one embodiment, peptides are chemically or recombinantly synthesized as oligopeptides derived from the amino acid sequence of iNOS or SSB. Alternatively, iNOS or SSB fragments are produced by digestion of native or recombinantly produced polypeptides by, for example, using a protease, e.g., trypsin, thermolysin, chymotrypsin, or pepsin. Computer analysis (using commercially available software, e.g. MacVector, Omega, PCGene, Molecular Simulation, Inc.) is used to identify proteolytic cleavage sites.

The peptide can also incorporate any number of natural amino acid conservative substitutions, insertions or deletions as long as such substitutions, insertions or deletions also do not substantially alter the peptide's structure and/or activity. Examples of conservative substitutions are shown in Table 1 under the heading of “exemplary substitutions”.

In addition, the skilled person can readily detect variants of peptides using amino acid sequence alignments and comparisons. Alignments and amino acid sequence comparisons are routinely performed in the art, for example, by using the BLAST program or the CLUSTAL W program.

TABLE 1
Exemplary substitutions.

| Original | Exemplary |
|---|---|
| Residue | Substitutions |
| Ala (A) | Val; Leu; Ile; Gly |
| Arg (R) | Lys |
| Asn (N) | Gln; His |
| Asp (D) | Glu |
| Cys (C) | Ser |
| Gln (Q) | Asn; His |
| Glu (E) | Asp |
| Gly (G) | Pro, Ala |
| His (H) | Asn; Gln |
| Ile (I) | Leu; Val; Ala |
| Leu (L) | Ile; Val; Met; Ala; Phe |
| Lys (K) | Arg |
| Met (M) | Leu; Phe |
| Phe (F) | Leu; Val; Ala |
| Pro (P) | Gly |
| Ser (S) | Thr |
| Thr (T) | Ser |
| Trp (W) | Tyr; Phe |
| Tyr (Y) | Trp; Phe |
| Val (V) | Ile; Leu; Met; Phe, Ala |

As with variants of peptides, routine experimentation will determine whether a peptide or mimetic thereof is within the scope of the invention, i.e., that its structure and/or function is not substantially altered.

The terms “mimetic”, “peptidomimetic” and “mimic” as used herein refer to a synthetic chemical compound, that has substantially the same structural and/or functional characteristics of the peptides, e.g., peptides of the invention derived from the amino acid sequence of iNOS or SSB. The mimetic can be entirely composed of synthetic, non-natural analogues of amino acids, or, may be a chimeric molecule of partly natural amino acid residues and partly non-natural analogs of amino acids.

A peptide may be characterized as a mimetic when all or some of its residues are joined by chemical means other than natural peptide bonds. Individual mimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, but not limited to, ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) In: Chemistry and BioChemistry of Amino Acids, Peptides and Proteins, Vol. 7, pp 267-357, “Peptide Backbone Modifications,” Marcell Dekker, NY).

A mimetic also can be a peptide-like molecule which contains, for example, an amide bond isostere such as a retro-inverso modification; reduced amide bond; methylenethioether or methylene-sulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. Retro-inverso modification of naturally occurring peptides involves the synthetic assembly of amino acids with α-carbon stereochemistry opposite to that of the corresponding L-amino acids, i.e., D- or D-allo-amino acids in inverse order to the native peptide sequence. A rerto-inverso analogue, thus, has reversed termini and reversed direction of peptide bonds, while essentially maintaining the topology of the side chains as in the native peptide sequence. One skilled in the art understands that these and other mimetics are encompassed within the meaning of the term “mimetic” as used herein.

The peptide or mimetic thereof of the invention may be any length so long as it binds to iNOS or SSB and blocks binding of SSB to iNOS. For example the peptide of the invention may be 30, 25, 20, 19, 18, 17, 16, 15, 14, 13, 12, 11 or fewer residues in length, or even shorter, for example, the peptide or mimetic thereof may be 10, 9, 8 or fewer residues in length.

In addition, the compounds useful for the invention, preferably peptides or mimetics as described herein may be fused to a cell penetrating agent, for example a cell-penetrating peptide, or a marcrophage targeting agent. Cell penetrating peptides include Tat peptides, Penetratin, short amphipathic peptides such as those from the Pep- and MPG-families, oligoarginine and oligolysine. Other cell penetrating agents include lipids such as a straight chain fatty acid.

### Antibodies

In one embodiment, the compound which binds to SSB or iNOS and which inhibits binding of SSB to iNOS is an antibody.

The term “antibody” as used herein includes polyclonal antibodies, monoclonal antibodies, bispecific antibodies, diabodies, triabodies, heteroconjugate antibodies, chimeric antibodies including intact molecules as well as fragments thereof, and other antibody-like molecules. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain. A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric (Morrison et al., 1984) or humanized (Jones et al., 1986). As used herein the term “antibody” includes these various forms. Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies for use in the methods of the present invention can be readily made.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

In one embodiment, the antibodies have the capacity for intracellular transmission. Antibodies which have the capacity for intracellular transmission include antibodies such as camelids and llama antibodies, shark antibodies (IgNARs), scFv antibodies, intrabodies or nanobodies, for example, scFv intrabodies and VHH intrabodies. Such antigen binding agents can be made as described by Harmsen and De Haard, 2007; Tibary et al., 2007; Muyldermans, 2001; and references cited therein. Yeast SPLINT antibody libraries are available for testing for intrabodies which are able to disrupt protein-protein interactions (see for example, Visintin et al., 2008a and Visintin et al, 2008b for methods for their production). Accordingly, in one embodiment, scFv intrabodies which are able to interfere with a protein-protein interaction are used in the methods of the invention. Such agents may comprise a cell-penetrating peptide sequence or nuclear-localizing peptide sequence such as those disclosed in Constantini et al., 2008. Also useful for in vivo delivery are Vectocell or Diato peptide vectors such as those disclosed in De Coupade et al., 2005 and Meyer-Losic et al., 2006.

In addition, the antibodies may be fused to a cell penetrating agent, for example a cell-penetrating peptide. Cell penetrating peptides include Tat peptides, Penetratin, short amphipathic peptides such as those from the Pep- and MPG-families, oligoarginine and oligolysine. In one example, the cell penetrating peptide is also conjugated to a lipid (C6-C18 fatty acid) domain to improve intracellular delivery (Koppelhus et al., 2008). Examples of cell penetrating peptides can be found in Howl et al., 2007 and in Deshayes et al., 2008. Thus, the invention also provides the therapeutic use of antibodies fused via a covalent bond (e.g. a peptide bond), at optionally the N-terminus or the C-terminus, to a cell-penetrating peptide sequence.

Although not essential, the antibody may bind specifically to iNOS or SSB. The phrase “bind specifically,” means that under particular conditions, the antibody binds iNOS or a SSB polypeptide and does not bind to a significant amount to other proteins or carbohydrates. Specific binding to iNOS or SSB under such conditions may require an antibody that is selected for its specificity. A variety of immunoassay formats may be used to select antibodies specifically immunoreactive with iNOS or SSB. For example, solid-phase ELISA immunoassays are routinely used to select antibodies specifically immunoreactive with a protein or carbohydrate. See Harlow and Lane (1988) Antibodies, a Laboratory Manual, Cold Spring Harbor Publications, New York, for a description of immunoassay formats and conditions that can be used to determine specific immunoreactivity.

In one embodiment, the antibody binds to a region of iNOS which binds SSB. For example, the antibody may bind within a sequence of amino acids of iNOS as provided in any one of SEQ ID NOs:1-22.

In another embodiment, the antibody binds to a region of SSB which binds iNOS. By way of non-limiting example, the antibody may bind within a sequence of amino acids of SSB as provided in any one of SEQ ID NOs:64 to 82, and/or the antibody binds to one or more of residues E55, N56, R68, P70, A72, R100, G101, T102, H103, Y120, L123, L124, L125, S126, N127, S128, V206, W207 or G208 of SSB-2, or to corresponding residues in homologous or orthologous SSB proteins as described herein, or to an epitope which comprises one or more of said residues.

### Modulating the Level of SSB in a Cell

The skilled person will appreciate from the teachings of the present application that increasing SSB activity in a cell will result in a decrease in the level of iNOS in the cell. It may be desirable to decrease the level of iNOS in a cell, for example, in a subject suffering from sepsis-induced lung injury, asthma, septic shock, excessive inflammation or excessive cytokine production. A polypeptide comprising SSB, or a polypeptide comprising at least the SPRY domain and SOCS box of SSB, when administered to a cell will bind to iNOS and associate with the E3 ligase complex, thus resulting in the polyubiquitination and degradation of iNOS. Accordingly, in one embodiment, the method comprises administering to a cell an isolated polynucleotide encoding a polypeptide comprising the SPRY domain and SOCS box of SSB, or an isolated polypeptide comprising the SPRY domain and SOCS box of SSB, whereby the level of iNOS in the cell is reduced. In one embodiment, the isolated polynucleotide may encode, or the polypeptide may comprise, full-length SSB.

In some instances it is desirable to reduce the level of SSB in a cell so as to increase the level of iNOS in the cell, for example when treating an infection in a subject. Thus, in one embodiment, the level of iNOS in a cell is modulated with a polynucleotide which reduces the level of SSB activity in the cell. Such polynucleotides include antisense polynucleotides, catalytic polynucleotides, microRNAs, and double-stranded RNA molecules such as siRNAs and shRNAs.

### Antisense Polynucleotides

The term “antisense polynucleotide” shall be taken to mean a DNA or RNA, or combination thereof, molecule that is complementary to at least a portion of a specific mRNA molecule encoding a polypeptide and capable of interfering with a post-20 transcriptional event such as mRNA translation. The use of antisense methods is well known in the art (see for example, G. Hartmann and S. Endres, Manual of Antisense Methodology, Kluwer (1999)).

An antisense polynucleotide useful for the invention will hybiidize to a target polynucleotide under physiological conditions. As used herein, the term “an antisense polynucleotide which hybridises under physiological conditions” means that the polynucleotide (which is fully or partially single stranded) is at least capable of forming a double-stranded polynucleotide with mRNA encoding a protein, in a cell.

Antisense molecules may include sequences that correspond to the structural genes or for sequences that effect control over the gene expression or splicing event. For example, the antisense sequence may correspond to the targeted coding region of the target gene, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Perriman et al., 1992) and the hairpin ribozyme (Shippy et al., 1999).

The ribozymes useful for this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides useful for the invention should also be capable of hybridizing a target nucleic acid molecule under “physiological conditions”, namely those conditions within a cell (especially conditions in an animal cell such as a human cell).

### RNA Interference

The terms “RNA interference”, “RNAi” or “gene silencing” refer generally to a process in which a double-stranded RNA molecule reduces the expression of a nucleic acid sequence with which the double-stranded RNA molecule shares substantial or total homology. However, it has more recently been shown that RNA interference can be achieved using non-RNA double stranded molecules (see, for example, US 20070004667).

The methods of the present invention utilise nucleic acid molecules comprising and/or encoding double-stranded regions for RNA interference. The nucleic acid molecules are typically RNA but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at. least 90% and more preferably at least 95%, 96%, 97%, 98%, 99%, or 100%. The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as 30 post transcriptional gene silencing, translational inhibition, or epigenetics. For example, siRNA molecules as described herein can be used to epigenetically silence genes at both the post-transcriptional level or the pre-transcriptional level. In a non-limiting example, epigenetic regulation of gene expression by siRNA molecules as described herein can result from siRNA mediated modification of chromatin structure to alter gene expression.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity.

Included shRNAs are dual or bi-finger and multi-finger hairpin dsRNAs, in which the RNA molecule comprises two or more of such stem-loop structures separated by single-stranded spacer regions.

Once designed, the nucleic acid molecules comprising a double-stranded region can be generated by any method known in the art, for example, by in vitro transcription, recombinantly, or by synthetic means.

Modifications or analogs of nucleotides can be introduced to improve the properties of the nucleic acid molecules. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

Examples of RNAi molecules that can be used to reduce SSB activity are described in Wang et al., 2005.

microRNA

MicroRNA regulation is a specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute 30 proteins that exert sequence-specific gene repression (see, for example, Millar and Waterhouse, 2005; Pasquinelli et al., 2005; Almeida and Allshire, 2005).

### Compositions and Administration

In certain embodiments, the present invention provides compositions comprising a compound of the invention and a suitable carrier or excipient. In one embodiment, the composition is a pharmaceutical composition comprising a pharmaceutically acceptable carrier. The compounds, for example peptides or mimetics thereof, are incorporated into pharmaceutical compositions suitable for administration to a mammalian subject, e.g., a human or a dog. Such compositions typically comprise the “active” composition (e.g., the peptide or mimetic) and a “pharmaceutically acceptable carrier”. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral (e.g., intravenous, intradermal, subcutaneous, intramuscular, intraperitoneal, intrathecal), mucosal (e.g., oral, rectal, intranasal, buccal, vaginal, respiratory), enteral (e.g., orally, such as by tablets, capsules or drops, rectally) and transdermal (topical, e.g., epicutaneous, inhalational, intranasal, eyedrops, vaginal). Solutions or suspensions used for parenteral, intradermal, enteral or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (e.g., glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid and the like. In many cases, it is preferable to include isotonic agents, for example, sugars, polyalcohols such as manitol, sorbitol, sodium chloride in the composition. Prolonged absorption of the injectable compositions is brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound is incorporated with excipients and used in the form of tablets, troches, or capsules. Oral compositions are also prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as, microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. For transdermal administration, the active compounds are formulated into ointments, salves, gels, or creams as generally known in the art.

A pharmaceutically acceptable vehicle is understood, to designate a compound or a combination of compounds entering into a pharmaceutical composition which does not cause side effects and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen.

### Screening Assays

One embodiment of the present invention relates to the use of SSB, or an iNOS binding fragment thereof, or iNOS, or an SSB binding fragment thereof, in a method for screening candidate compounds in vitro or in vivo for compounds that modulate the binding of SSB to iNOS and which may be useful for modulating the level of iNOS in a cell.

By a “candidate compound” is meant an agent to be evaluated for the ability to bind to SSB or iNOS and reduce binding of SSB to iNOS. Candidate compounds may include, for example, peptides, polypeptides, antibodies, mimetics, synthetic organic molecules, naturally occurring organic molecules, nucleic acid molecules such as aptamers, peptide nucleic acid molecules, and components and derivatives thereof.

In certain embodiments, combinatorial libraries of potential inhibitors will be screened for an ability to bind to the protein sequence of SSB or iNOS and modulate the ability of SSB to bind iNOS.

Conventionally, new chemical entities with useful properties are generated by identifying a chemical compound (called a “lead compound”) with some desirable property or activity, e.g., reducing binding, creating variants of the lead compound, and evaluating the property and activity of those variant compounds. Often, high throughput screening (HTS) methods are employed for such an analysis.

In one embodiment, high throughput screening methods involve providing a library containing a large number of candidate compounds. Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., 1994).

Preparation and screening of combinatorial chemical libraries is well known to those of skill in the art. Such combinatorial chemical libraries include, but are not limited to, peptide libraries, peptoids, encoded peptides, random bio-oligomers, nonpeptidal mimetics, analogous organic syntheses of small compound libraries, nucleic acid libraries, peptide nucleic acid libraries, antibody libraries, carbohydrate libraries and small organic molecule libraries.

Compounds which bind to SSB or iNOS may be identified and isolated by methods known to those of skill in the art. Examples of methods that may be used to identify such binding compounds are the yeast-2-hybrid screening, surface Plasmon resonance, high-resolution NMR, phage display, affinity chromatography, expression cloning, immunoprecipitation and GST pull downs coupled with mass spectroscopy.

Surface Plasmon Resonance (SPR) or Biomolecular Interaction Analysis (BIA; e.g., Biacore) detects biospecific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules.

Information from SPR can be used to provide an accurate and quantitative measure of the equilibrium dissociation constant (Kd), and kinetic parameters, including kₒₙ and k_{off}, for the binding of a molecule to a target. Such data can be used to compare different molecules. Information from SPR can also be used to develop structure-activity relationships (SAR). For example, the kinetic and equilibrium binding parameters of different peptides can be evaluated. Variant amino acids at given positions can be identified that correlate with particular binding parameters, e.g., high affinity and slow k_{w}. This information can be combined with structural modeling (e.g., using homology modeling, energy minimization, or structure determination by x-ray crystallography or NMR). As a result, an understanding of the physical interaction between the peptide and its target can be formulated and used to guide other design processes.

The assays to identify modulators of SSB binding to iNOS may be amenable to high throughput screening. High throughput assays for the presence, absence, quantification, or other properties of particular protein products are well known to those of skill in the art. Similarly, binding assays and reporter gene assays are similarly well known. Thus, e.g., U.S. Pat. No. 5,559,410 discloses high throughput screening methods for proteins, while U.S. Pat. Nos. 5,576,220 and 5,541,061 disclose high throughput methods of screening for ligand/antibody binding.

In addition, high throughput screening systems are commercially available. These systems typically automate entire procedures, including all sample and reagent pipetting, liquid dispensing, timed incubations, and final readings of the microplate in detectors) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

Standard solid-phase ELISA assay formats are also useful for identifying antagonists of protein-protein interaction. In accordance with this embodiment, one of the binding partners, e.g. SSB, is immobilized on a solid matrix, such as, for example an array of polymeric pins or a glass support. Conveniently, the immobilized binding partner may be a fusion polypeptide comprising, for example, Glutathione-S-transferase, wherein the GST moiety facilitates immobilization of the protein to the solid phase support. The second binding partner (e.g. iNOS or a SSB binding fragment thereof) in solution is brought into physical relation with the immobilized protein to form a protein complex, which complex is then detected by methods known in the art. Alternatively, Histidine-tagged protein complexes can be detected by their binding to nickel-NTA resin, or FLAG=labeled protein complexes detected by their binding to FLAG M2 Affinity Gel. It will be apparent to the skilled person that the assay format described herein is amenable to high throughput screening of samples, such as, for example, using a microarray of bound peptides or fusion proteins.

### EXAMPLES

### Example 1

### Identification of iNOS as a Potential SSB Binding Partner

The SPRY domains of murine SSB-1 and SSB-2′ have previously been shown to interact with a peptide motif [DE]-[IL]-N-N-N-[LN] (SEQ ID NO:36) present in Drosophila VASA and human PAR-4 (Woo et al., 2006). While the motif responsible for SSB binding is present in human PAR-4, the motif is absent in murine PAR-4, and indeed murine PAR-4 does not bind SSB proteins (data not shown). Likewise, the VASA localization function of GUSTAVUS (SEQ ID NO:71) in Drosophila (Styhler et al., 2002) does not seem to be shared by its mouse or human homolog proteins, SSB-1 and SSB-4, since neither murine or human VASA contains the DINNN sequence responsible for GUSTAVUS binding. The lack of conservation of the binding sequences between species strongly suggests that neither PAR-4 nor VASA are the physiological targets of the SSB proteins. The present inventors therefore sought to identify other SSB-2 binding proteins as candidate physiological targets.

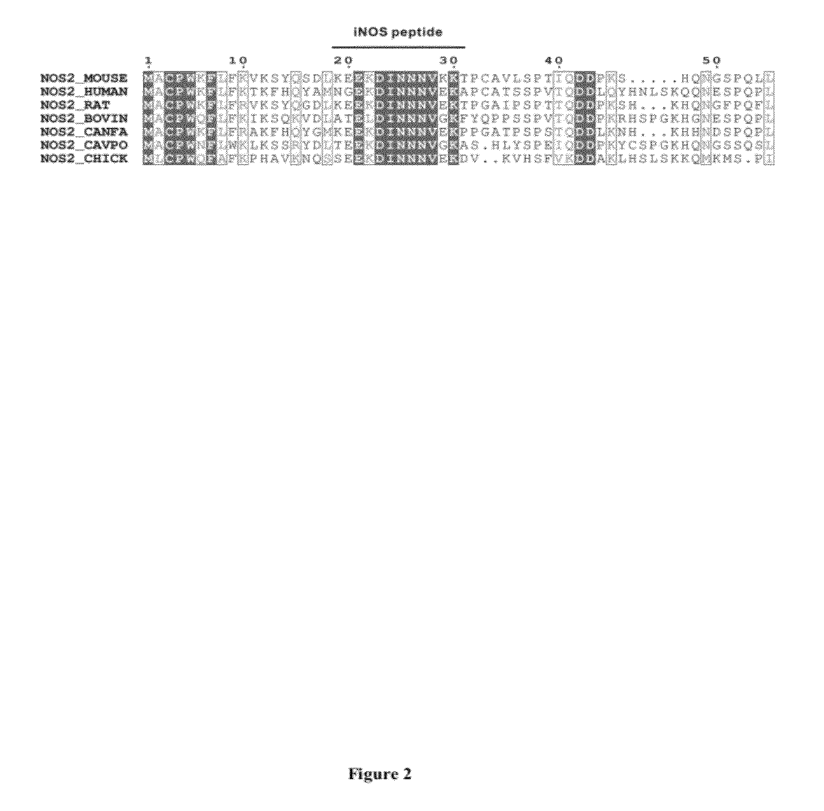

A sequence analysis using ScanProsite (de Castro et al., 2006) identified 11 mouse proteins and 16 human proteins that contained the [DE]-[IL]-N-N-N (SEQ ID NO:36) sequence, and included inducible nitric oxide synthase (iNOS or NOS2). The DINNN motif is located in the N-terminal region of mouse iNOS prior to the first structured domain, the oxygenase domain (amino acids 23-27 of mouse iNOS). This motif and certain flanking residues are conserved in iNOS sequences from different species (FIG. 2), including human, mouse, bovine, chicken and goldfish, whereas neuronal nitric oxide synthase (nNOS or NOS1) or endothelia nitric oxide synthase (eNOS or NOS3) do not contain this motif (data not shown). The N-terminal region of iNOS is predicted to be intrinsically disordered using the programs FoldInex (Prilusky et. al., 2005) and IUPred (Dostzanyi et al., 2005) (data not shown), further suggesting that this region is accessible for SSB binding.

### Example 2

### SSB SPRY Domain Interacts with iNOS N-terminal Sequence

### Materials and Methods

cDNA Cloning

Oligonucleotides were designed which were specific to individual mouse Spsb 10 genes. cDNA clones covering the entire coding region of murine SSB-1 to −4 were isolated by overlapping PCR from commercially available cDNA libraries or a bacterial artificial chromosome (mouse BAC 6). Constructs encoding proteins with an N-terminal Flag epitope tag (DYKDDDDK (SEQ ID NO:37)) were generated by PCR to give fragments with in-frame Asc I and Mlu I restriction enzyme sites at the N- and C-termini, respectively, and sub-cloned into the mammalian expression vector pEF-FLAG-I, a derivative of the mammalian expression vector pEF-BOS (Mizushima and Nagata, 1990). SSB-2 point mutants were generated using the PCR-based technique, splicing by overlap extension (Horton et al., 1989).

### Protein Production

The construct used for expression of recombinant murine SSB-2 protein included almost all the native sequence of mouse SSB-2 except for the SOCS box and the first eleven residues (residues 12-224, SWISS-PROT accession number 088838). This sequence, together with six residues at the N-terminus (GSSARQ (SEQ ID NO:38), numbered 6-11) and seven at the C-terminus (TRRIHRD (SEQ ID NO:39), numbered 225-231), both originating from the vector, gave a construct of 226 residues in total. This was expressed as a GST fusion protein in BL21 (DE3) E. coli. For ITC and co-precipitation experiments, bacteria were grown in L-broth. For NMR analysis, bacteria were grown in M9 minimal media supplemented with ¹⁵N NH₄Cl (99%, 1 g L−1). The GST fusion protein was purified from clarified cell lysates using Glutathione Sepharose 4B (Amersham Biosciences) then cleaved in situ using thrombin (Roche). The cleaved protein was then concentrated and further purified by gel filtration using a Superdex 200 column (Amersham Biosciences).

Isothermal Titration calorimetry (ITC)

Wild-type and mutant iNOS peptides (Table 2), corresponding to Lys19-Thr31 of mouse iNOS, were synthesized by GL Biochem (Shanghai) Ltd. These peptides were N-terminal acetylated and C-terminal amidated. All ITC measurements were carried out at 25° C. using a Microcal omega VP-ITC (Microcal Inc., Northampton, Mass.). SSB-2ΔSB was dialysed against buffer (100 mM TrisHCl, 150 mM NaCl, pH 8.0), and wild-type and mutant iNOS N-terminal peptides were prepared in the same buffer from 5 mM stocks. Solutions of 5 to 10 μM SSB-2ΔSB in the cell were titrated by injection of a total of 290 μL of 50-200 μM of iNOS peptides. Data analysis was performed using the evaluation software, MicroCal Origin version 5.0. All curves were fitted using the nonlinear least-squares fitter and the “One Set of Sites” model.

### NMR Spectroscopy

NMR spectra were recorded on an Avance 500 spectrometer equipped with a cryoprobe. The ¹H chemical shifts were referenced indirectly to DSS at 0 ppm via the H₂O signal, and the ¹³C and ¹⁵N chemical shifts were referenced indirectly using absolute frequency ratios (Wishart et al., 1995). Spectra were processed using Topspin version 1.3 (Bruker Biospin) and analysed using XEASY, version 1.3. ¹⁵N-labelled SSB-2ΔSB Sample for NMR analysis were prepared in H₂O containing 5% ²H₂O, 10 mM sodium phosphate, 50 mM sodium chloride, 2 mM EDTA, 2 mM DTT and 0.02% (w/v) sodium azide at pH 7.0. Two-dimensional ¹H-¹⁵N HSQC spectrum of a 0.1 mM ¹⁵N-labelled SSB-2ΔSB sample was recorded and 22° C. using a data matrix size of 2048×256 and with 128 scans per t1 increment. The spectral widths were 13.5 ppm for ¹H and 40.0 ppm for ¹⁵N; carrier frequencies were 4.7 ppm for ¹H and 118 ppm for ¹⁵N. Unlabelled iNOS N-terminal peptide (wild-type) were then titrated into the ¹⁵N-labelled SSB-2ΔSB sample, and ¹H-¹⁵N HSQC spectra recorded at ¹⁵N-labelled SSB-2ΔSB:iNOS peptide ratios of 1:0.5, 1:1, and 1:1.5.

### Results

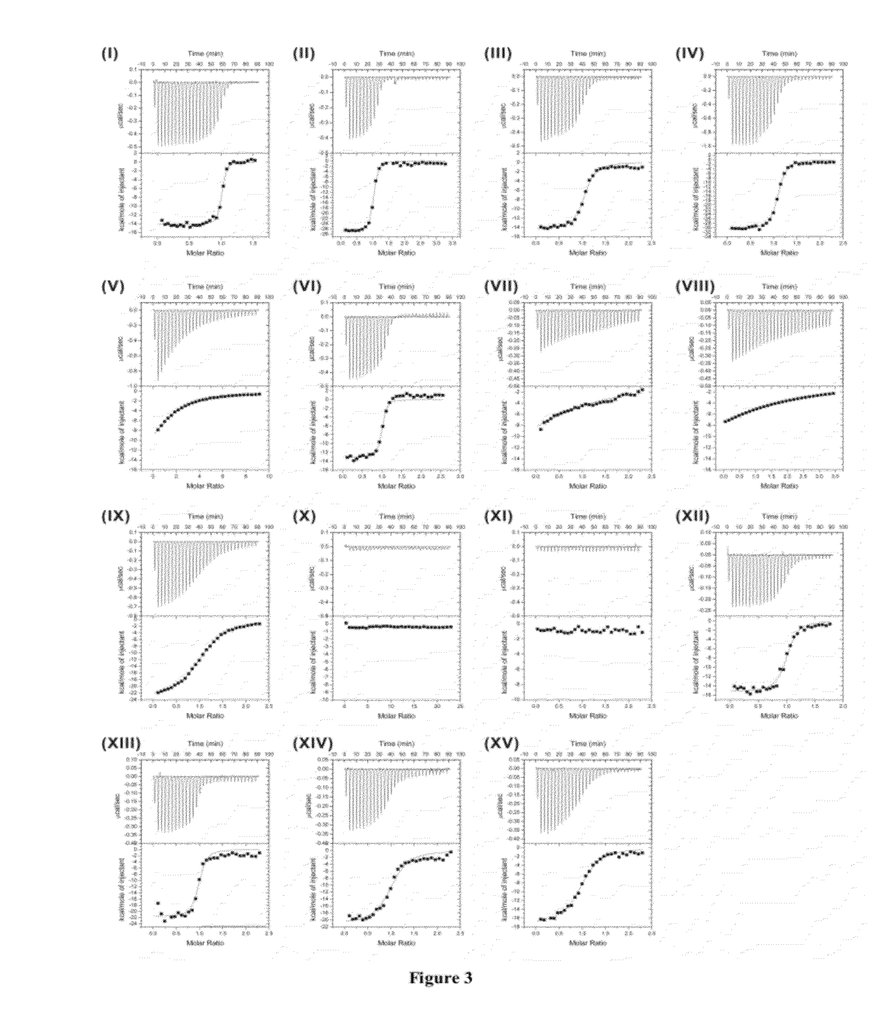

To determine whether the SSB-2-SPRY domain could interact with the sequence identified by the database searches, a series of peptides including the wild-type and various mutant sequences corresponding to amino acids 19-31 of murine iNOS, were synthesized and binding affinities for the SSB-2-SPRY domain (residues 12-224; SSB-2ΔSB) measured using isothermal titration calorimetry (ITC) (Table 2, FIG. 3).

The wild-type iNOS peptide bound SSB-2 with high affinity (K_{D}=13 nM). Mutation of Asp27 to Ala within the iNOS peptide dramatically reduced the binding affinity, indicating that this residue makes a major contribution to binding and is consistent with the structural requirements previously reported for the GUSTAVUS-VASA interaction (Woo et al., 2006). Additional conserved residues flanking the DINNN (SEQ ID NO:36) sequence (Lys22, Val28 and Lys30) also contribute to the interaction, as alanine substitutions of these residues had 2- to 4-fold lower SSB-2 binding affinities than the wild-type peptide. Indeed, when these three residues were mutated to Ala simultaneously, SSB-2 binding affinity was decreased by ˜24-fold, suggesting that the SSB binding sequence in iNOS is more extensive than reported for VASA:GUSTAVUS or PAR-4:SSB binding (Woo et al., 2006). Mutation of Glu21 had no effect on SSB-2:iNOS peptide binding. Tyrosine 120 in SSB-2 has previously been shown to be critical for interaction with Par-4 (Masters et al., 2006). Binding of an SSB-2-SPRY domain in which tyrosine 120 had. been mutated to alanine (Y120A-SSB-2ΔSB) was assessed by ITC. The iNOS peptide bound Y120A-SSB-2ΔSB with ˜5000-fold reduced affinity, evidence that Tyr120 in SSB-2 is critical for binding to the iNOS peptide (Table 2).

TABLE 2
ITC analysis of the interaction between the
SSB-2 SPRY domain and wild-type or mutant
iNOS N-terminal peptides.

| Peptide | Sequence | KD (nM)^{a} |
|---|---|---|
| I | Ac-KEEKDINNNVKKT-NH₂ | 13.3 ± 3.0 |
| | (SEQ ID NO: 3) | |
| II | Ac-KEAKDINNNVKKT-NH₂ | 14.0 ± 3.0 |
| | (SEQ ID NO: 4) | |
| III | Ac-KEEADINNNVKKT-NH₂ | 127 ± 23 |
| | (SEQ ID NO: 5) | |
| IV | Ac-KEAADINNNVKKT-NH₂ | 65.4 ± 7.4 |
| | (SEQ ID NO: 6) | |
| V | Ac-KEEKAINNNVKKT-NH₂ | 21600 ± 750 |
| | (SEQ ID NO: 7) | |
| VI | Ac-KEEKDANNNVKKT-NH₂ | 23.5 ± 9.9 |
| | (SEQ ID NO: 8) | |
| VII | Ac-KEEKDIANNVKKT-NH₂ | 17200 ± 7400 |
| | (SEQ ID NO: 9) | |
| VIII | Ac-KEEKDIQNNVKKT-NH₂ | 40500 ± 7200 |
| | (SEQ ID NO: 10) | |
| IX | Ac-KEEKDINANVKKT-NH₂ | 826 ± 20 |
| | (SEQ ID NO: 11) | |
| X | Ac-KEEKDINNAVKKT-NH² | ND^{b} |
| | (SEQ ID NO: 12) | |
| XI | Ac-KEEKDINNQVKKT-NH₂ | ND |
| | (SEQ ID NO: 13) | |
| XII | Ac-KEEKDINNNAKKT-NH₂ | 56.8 ± 11.2 |
| | (SEQ ID NO: 14) | |
| XIII | Ac-KEEKDINNNVKAT-NH₂ | 29.8 ± 12.2 |
| | (SEQ ID NO: 15) | |
| XIV | Ac-KEEKDINNNAKAT-NH₂ | 180 ± 39 |
| | (SEQ ID NO: 16) | |
| XV | Ac-KEEADINNNAKAT-NH₂ | 311 ± 37 |
| | (SEQ ID NO: 17) | |
| Y120A- | Ac-KEEKDINNNVKKT-NH₂ | 76300 ± 18000 |
| SSB-2ΔSB | (SEQ ID NO: 3) | |

^{a}Affinities are quoted as dissociation constants with the errors from the Origin-calculated association constants transferred as the same fractions of primary values.
^{b}ND: binding affinity was too low to be determined under these conditions.

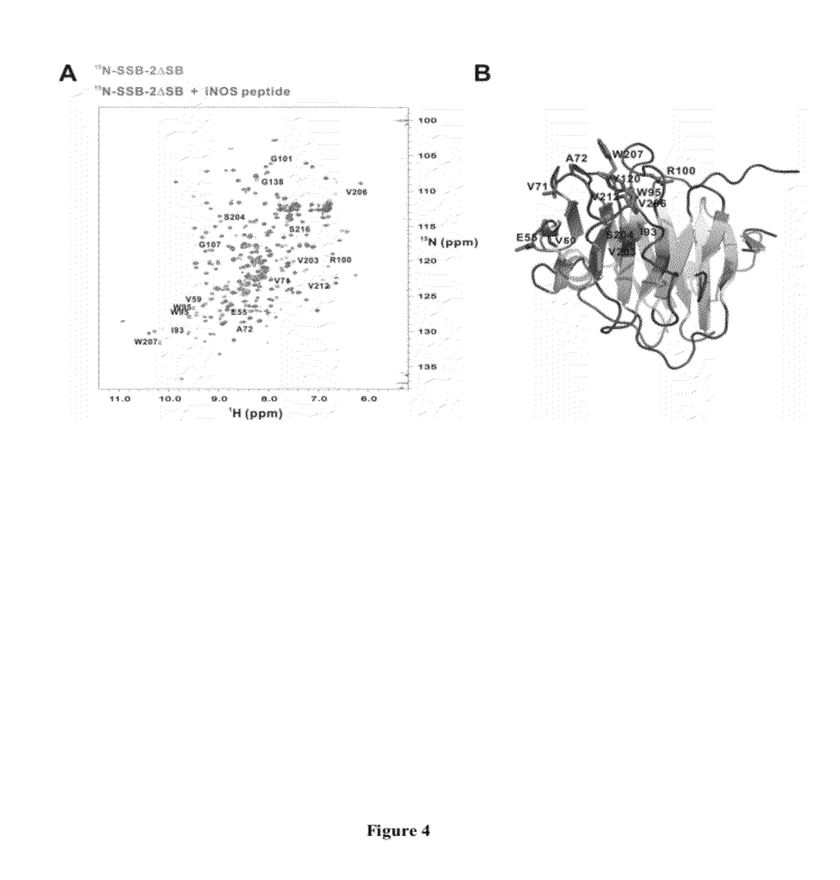

Nuclear magnetic resonance (NMR) spectroscopy was then used to further analyze the SSB-2:iNOS peptide interaction. Titration of the unlabeled wild-type iNOS N-terminal peptide into a ¹⁵N-labelled SSB-2ΔSB sample caused a gradual disappearance of the “free” set of SSB-2ΔSB crosspeaks and the simultaneous appearance of a “bound” set of cross-peaks in the ¹H-¹⁵N HSQC spectra (FIG. 4A). This was further confirmation that the iNOS peptide bound to SSB-2ΔSB and showed that the interaction was in the slow exchange regime on the NMR time scale. The residues that exhibited relatively larger chemical shift perturbations are found on a continuous surface on SSB-2 in the vicinity of Y120, V206, and W207, forming an iNOS peptide-binding site (FIG. 4B).

### Example 3

### SSB-1, 2, and 4 Interact with Full-length iNOS Protein

### Materials and Methods

### Antibodies

Rabbit polyclonal anti-SSB-2 antibodies have been described previously (Masters et al., 2005). Affinity-purified anti-SSB-2 antibodies were either conjugated to NHS-Sepharose at 1.5 mg/ml or biotinylated using sulfo-NHS-Biotin (Pierce, Rockford, Ill.) according to the manufacturer's instructions. Mouse monoclonal anti-iNOS antibody was obtained from BD Biosciences (Phaminogen) and mouse monoclonal anti-α-tubulin antibody from Sigma (Saint Louis, Mich.).

### Immunoprecipitation and Western Blot

Bone marrow-derived macrophages were generated as described and re-plated at 1.0×10⁶ cells/well on 6-well plates (Costar) in DME containing 10% FCS and 20% L-cell conditioned media. Cells were incubated with IFNγ and LPS as described, and lysed in KALB lysis buffer (Nicholson et al., 1995) containing protease inhibitors (Complete Cocktail tablets, Roche), 1 mM phenylmethylsulphonyl fluoride, 1 mM Na₃VO₄ and 1 mM NaF. Proteins were immunoprecipitated using anti-Flag antibody conjugated to Sepharose (M2; EASTMAN KODAK). Proteins were separated by sodium dodecyl sulphatepolyacrylamide gel electrophoresis (SDS-PAGE) under reducing conditions and electrophoretically transferred to Biotrace PVDF membranes (Pall Corp. Ann Arbor, Mich.). Membranes were blocked overnight in 10% w/v skim milk and incubated with primary antibody for 2 h. Antibody binding was visualized with either peroxidase-conjugated goat anti-rat immunoglobulin (Southern Biotech) or peroxidase-conjugated sheep anti-rabbit immunoglobulin (Chemicon, Melbourne, Australia), and the enhanced chemiluminescence (ECL) system (Amersham, Little Chalfont, Buckinghamshire, UK). To re-blot, the membrane was first stripped of antibodies in 0.1 M glycine, pH 2.9.

### Generation of Bone Marrow-Derived Macrophages (BMDM)

Murine bone marrow macrophages were derived by culture of whole bone marrow in Dulbecco's modified Eagle's medium (DMEM) supplemented with 100 U/ml penicillin, 0.1 mg/ml streptomycin, 10% fetal bovine serum (FBS) and 20% L-cell conditioned medium as a source of macrophage colony stimulating factor (M-CSF) (Wormald et al., 2006). FACS analysis confirmed that following six days in culture >95% of cells were positive for CD11b expression (Mac-1).

### Transient Transfection of 293T Cells

293T cells (DuBridge et al., 1987) were maintained in Dulbecco's Modified Eagles Medium (DMEM) supplemented with 100 U/ml penicillin, 0.1 mg/ml streptomycin and 10% foetal bovine serum (Sigma, St Louis Mo.). Cells were transiently transfected using FuGene6 Reagent (Roche, Mannheim, Germany) according to the manufacturer's instructions.

### Results

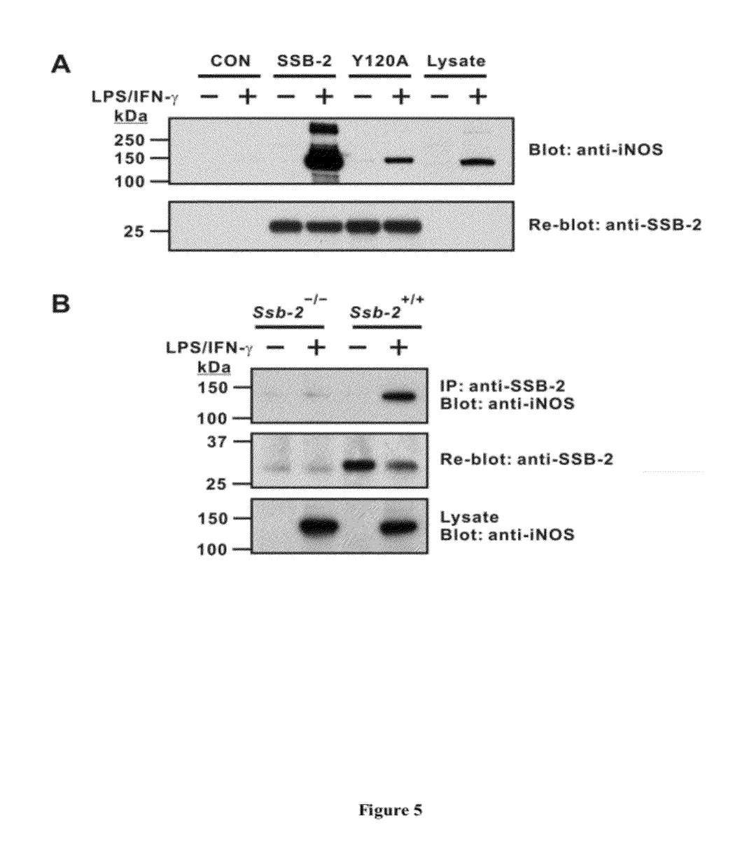

Whilst high affinity binding to the iNOS peptide was encouraging, it was important to confirm that SSB-2 was able to bind iNOS peptide within the context of full-length protein in its native conformation. iNOS protein was generated by LPS and IFN-γ-treatment of bone marrow-derived macrophages (BMDM). Cells were lysed and iNOS expressing lysates incubated with SSB-2ΔSB protein coupled to Sepharose beads. SSB-2-associated proteins were then separated by SDS-PAGE and iNOS detected by Western blot with specific antibodies. A strong interaction of the full-length iNOS protein was observed with SSB-2. In contrast, Y120A-SSB-2ΔSB co-precipitated only a minimal amount of iNOS, further confirming that Y120 is critical for the interaction (FIG. 5A). These results are consistent with our ITC data, and indicate that the iNOS-peptide binding site on SSB-2ΔSB as revealed by NMR is responsible for the binding of full-length iNOS protein.

To determine whether iNOS was a genuine candidate as a physiological target of SSB regulation, we next investigated whether the proteins co-existed in an endogenous complex. SSB-2 and iNOS proteins were co-immunoprecipitated from LPS/IFN-γ-stimulated C57BL/6 BMDM, but not from macrophages derived from SSB-2-deficient BMDM (Ssb-2^{-/-}), confirming an interaction between the endogenous proteins (FIG. 5B). Interestingly, the amount of immunoprecipitated SSB-2 protein was reduced in LPS/IFN-γ-activated BMDM compared to unstimulated controls; this was consistent with the regulation of SSB-2 mRNA (see below). Probing of lysates using specific antibody confirmed iNOS expression in cells derived from both wild-type and Ssb-2^{-/-} mice (FIG. 5B).

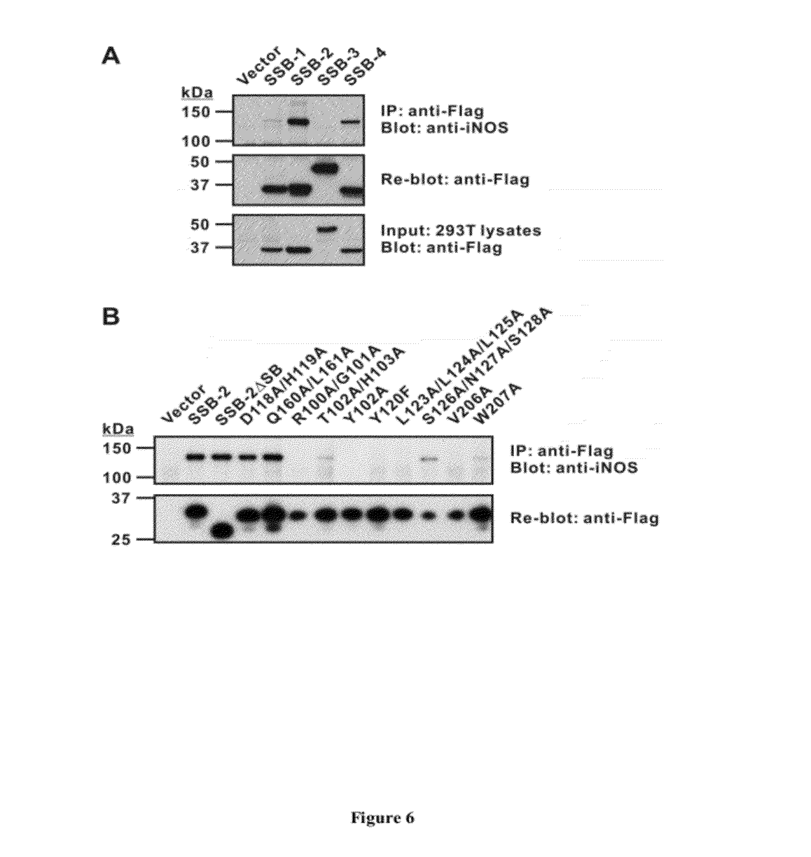

To investigate whether other SSB family members were able to interact with iNOS, 293T cells were transiently transfected with cDNA expressing SSB-1, SSB-2, SSB-3 or SSB-4 with an N-terminal Flag-epitopetag. 293T cells were lysed and mixed with iNOS-expressing lysates derived from BMDM. SSB proteins were immunoprecipitated using anti-Flag antibodies coupled to Sepharose and association of iNOS analysed by Western blot. SSB-2 and SSB-4, but not SSB-3 were able to co-precipitate iNOS protein. In comparison to SSB-2 and SSB-4, SSB-1 bound iNOS quite weakly, despite equivalent 30 expression levels of SSB protein as detected by anti-Flag blot of cell lysates, and suggesting that while SSB-1, -2 and -4 were able to interact with iNOS, binding affinity may differ between these family members (FIG. 6A).

To confirm the NMR results and further interrogate the SSB-2/iNOS binding interface, 293T cells were transiently transfected with cDNA expressing either SSB-2 or various SSB-2 mutants (Masters et al., 2006) and interaction with iNOS assessed as described earlier. Mutation of R100/G101, Y120, L123/L124/L125 or V206 to alanine or mutation of Y120 to phenylalanine completely abrogated SSB-2 interaction with iNOS. Mutation of T102/H103, S126/N127/S128 or W207 to alanine dramatically reduced SSB-2 interaction with iNOS, whilst mutation of either D118/H119 or Q160/L161 had no effect. Re-blot of the anti-Flag immunoprecipitates confirmed expression of the SSB-2 mutants (FIG. 6B). These results are consistent with the previously identified Par-4 binding site (Woo et al., 2006; Masters et al., 2006) and indicate that both Par-4 and iNOS have, at the least, an overlapping binding site on SSB-2.

### Example 4

### Expression of SSB-1 is Transiently Induced in Response to LPS

### Materials and Methods

### Real-Time Quantitative PCR (Q-PCR)

Bone marrow-derived macrophages were generated as described and replated at 1.0×10⁶ cells/well on 6-well plates (Costar) in DME containing 10% FCS and 20% L-cell conditioned media. The following day, triplicate cultures were incubated with murine IFNγ (10 ng/ml) and LPS (20 ng/ml), unless otherwise indicated. Total cellular RNA was isolated using the RNeasy kit (QIAGEN, Valencia, Calif.) and first strand cDNA synthesis performed using Superscript II RNASE H— reverse transcriptase (Invitrogen, Carlsbad, Calif.) according to the manufacturer's instructions. Real-time PCR was performed on an ABI Prism 7900HT sequence detection system (Applied Biosystems, Foster City, Calif.). Cycling conditions were as follows: initial denaturation (95° C. for 15 min), followed by 40 cycles of 94° C. for 15 s, 50° C. (SSB-1, -4), 60° C. (SSB-2, -3) or 49° C. (GAPDH) for 30 s and 72° C. for 15 s with a transition rate of 20° C./s and a single fluorescence measurement, melting curve program (60° C.-95° C., with a heating rate of 0.1° C./s and continuous fluorescence measurement) and a final cooling step to 40° C. All PCR reactions were performed using the QuantiTect SYBR Green PCR Kit (QIAGEN) in 10 μl reactions containing 0.5 μmol of forward and reverse primers, 5 μl of QuantiTect Master Mix and 4 μl of cDNA (diluted 1 in 5).

Primer sequences were as follows:

(SEQ ID NO: 23)
GAPDH (F): TTGTCAAGCTCATTTCCTGGT;
(SEQ ID NO: 24)
(R): TTACTCCTTGGAGGCCA TGTA;
(SEQ ID NO: 25)
SSB-1 (F): CGGGGACTCAAGGGTAAAA;
(SEQ ID NO: 26)
(R): AGGGGCTCAGGATCAAGTC;
(SEQ ID NO: 27)
SSB-2 (F): AAGAAGAGTGGAGGAACCACAAT;
(SEQ ID NO: 28)
(R): CAAAGGCAGAGTGGATA TTTGAC;
(SEQ ID NO: 29)
SSB-3 (F): GCAGCTCTAACTGGGCTATGACTC;
(SEQ ID NO: 30)
(R): ACAGGCACAGCACTGGGGATGGATG;
(SEQ ID NO: 31)
SSB-4 (F): GAGTGCTGTGTGGGGTCA;
(SEQ ID NO: 32)
(R): AGGGCTGAGCGGATGGAT;
(SEQ ID NO: 33)
iNOS (F): AGATCGAGCCCTGGAAGACC
(SEQ ID NO: 34)
(R): ATTAGCATGGAAGCAAAGAACAC

The specificity of the SYBR green reaction was assessed by melting point analysis and gel electrophoresis. mRNA levels were quantified from standard curves generated using dilutions of an oligonucleotide corresponding to the amplified fragment and using SDS 2.2 software (Applied Biosytems). Relative expression was determined by normalizing the quantity of the gene of interest to the quantity of glyceraldehyde 3-phosphate dehydrogenase (GAPDH). Each measurement was carried out in duplicate.

### Results

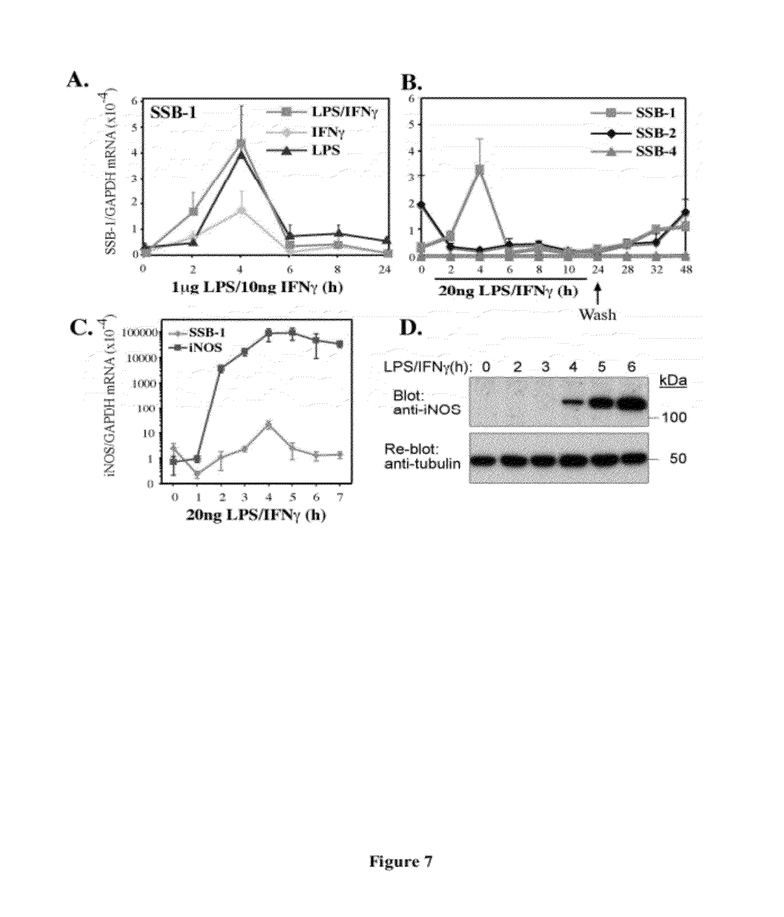

To better evaluate the effect of genetically deleting SSB-2 and given that multiple family members can potentially interact with iNOS, it was important to know whether SSB expression was regulated during iNOS induction. BMDM from C57BL/6 mice were treated with LPS/IFNγ, IFNγ or LPS alone. Cells were lysed at various times and total mRNA purified and reverse transcribed to cDNA for analysis of SSB and iNOS mRNA levels by Q-PCR. SSB-1 mRNA was rapidly induced in response. to either LPS/IFNγ or LPS alone, was maximal after 4 h stimulation and then decreased close to basal levels by 6 h. This effect appeared independent of LPS dose (FIGS. 7A and 7B). In contrast, SSB-2 mRNA levels had dropped modestly by 2 h, and then increased slightly with time and following removal of the stimulus (FIG. 7B). This reduction in mRNA level was accompanied by a modest change in protein expression (FIG. 5B). SSB-4 mRNA levels did not change and were barely detectable at baseline (FIG. 7B). It is possible that SSB-1 is induced as part of a negative feedback loop to regulate iNOS levels. The kinetics of iNOS mRNA and protein expression was therefore examined following LPS/IFNγ treatment. iNOS mRNA was induced 3000-fold within 2 h of treatment. In comparison, there was a delay in protein production with iNOS protein detected at 4 h and maximal at 6 h treatment (FIGS. 7C and 7D).

### Example 5

### SSB-2 and SSB-1 Regulate the Proteasomal Degradation of iNOS

### Materials and Methods

### Mice

Mice with a homozygous deletion of the Spsb-2 gene (Ssb-2^{-/-}) have been described previously (Masters et al., 2005) and were maintained on a C57BL/6 background. pUBc constructs containing the SSB-1 (Spsb-1) coding region with (a.a. 2-274) and without the SOCS box (a.a. 2-233) were generated to express SSB-1 with an N-terminal Flag epitope 30 under the ubiquitin C promoter. pUBc constructs containing the SSB-2 (Spsb-2) coding region with (a.a. 3-265) and without the SOCS box (a.a. 2-224) were generated to express SSB-2 with an N-terminal Flag epitope under the ubiquitin C promoter. Transgenic constructs were injected into C57BL/6 blastocysts followed by implantation into pseudopregnant C57BL/6 females. Progeny were screened by Southern blot for germ-line transmission of the transgene. Protein expression was confirmed by immunoprecipitation and Western blot with anti-Flag antibodies.

### Co-Precipitation Experiments

Bacterially expressed SSB-2 (SSB-2ΔSB; a.a.12-224) and SSB-2 protein in which tyrosine 120 had been mutated to alanine (Y120A-SSB-2ΔSB; a.a.12-224) were purified and conjugated to NHS sepharose as described previously (Masters et al., 2005). iNOS expressing BMDM lysates were pre-cleared with beads alone for 1 h and then incubated with SSB-2-coupled protein for 3 h at 4° C. iNOS interaction was then detected by SDS-PAGE and Western blot.

### Griess Assay

As a measure of nitric oxide production, 100 μl of culture supernatants were assayed for nitrite by reaction with 10 μl of Griess reagent A (1% sulfanilamide in 5% phosphoric acid) for 10 min at room temperature, followed by the addition of 10 μl Griess reagent B [0.14% N-(1-naphthyl)ethylenediamine dihydrochloride], and nitrite content determined essentially as described (Scott et al., 2000).

### Results

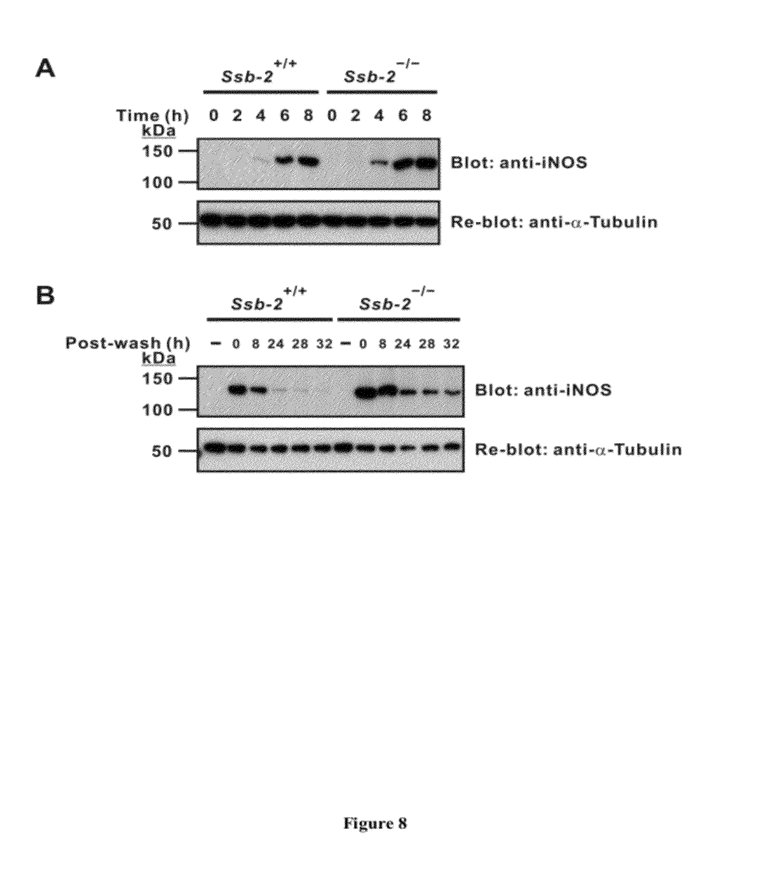

Although iNOS is known to be ubiquitinated and degraded via the proteasome in cell lines, the E3 ubiquitin ligase/s responsible have not been identified. To determine whether either the kinetics or magnitude of iNOS expression was altered in the absence of SSB-2, BMDM from SSB-2-null mice (Ssb-2^{-/-}) or wild-type littermates were stimulated with LPS/IFN-γ for various times, lysed and iNOS expression detected by Western blot. Although the initial kinetics of iNOS induction appeared to be the same in both wild-type and Ssb-2^{-/-} BMDM, slightly more iNOS protein was detected in Ssb-2^{-/-} BMDM after 4 h stimulation (FIG. 8A). A greater difference in iNOS expression between wild-type and Ssb-2^{-/-} BMDM was evident after the stimulus was removed and iNOS began to be degraded (FIG. 8B); these results are consistent with the decreased SSB-2 mRNA level during LPS/IFN-γ stimulation and the recovery of SSB-2 expression following removal of LPS/IFN-γ. Indeed, clearance of iNOS in wild-type BMDM was essentially complete at 24 h post-wash, whereas in Ssb-2^{-/-} BMDM, iNOS was still clearly visible after 32 h (FIG. 8B). The results indicate that while the initial kinetics of iNOS induction are unaltered in Ssb-2^{-/-} BMDM clearance of iNOS protein by the degradation machinery is impaired.

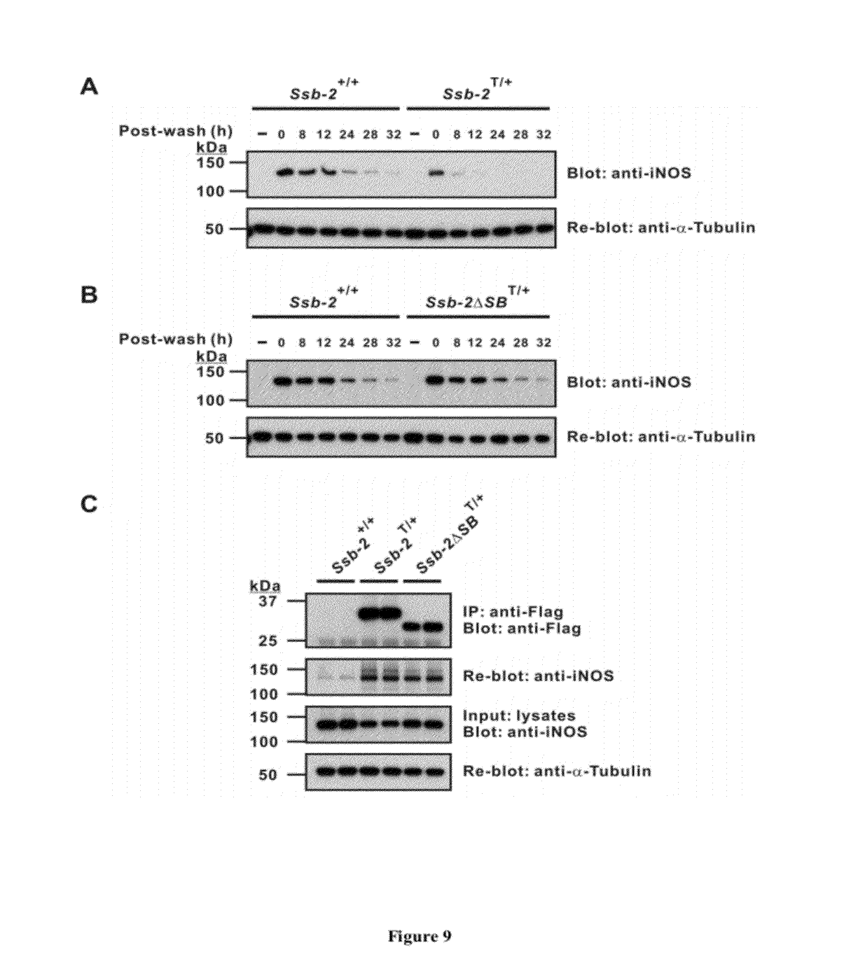

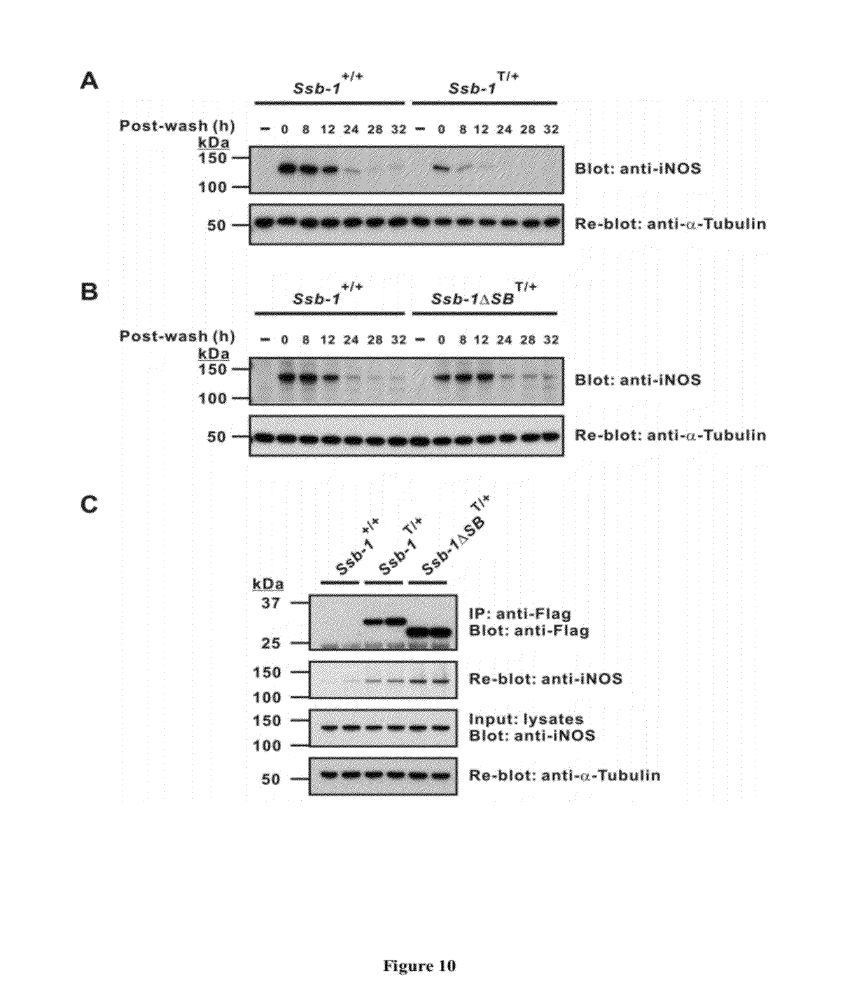

As genetic deletion of SSB-2 appeared to result in elevation of iNOS levels, it was tested whether artificially increasing SSB levels could correspondingly down-regulate iNOS expression. BMDM were generated from mice expressing either a Flag-tagged Ssb-1 or Ssb-2 transgene under a constitutive promoter (Ssb-1^{T/+}, Ssb-2^{T/+}) and from wild-type littermate control mice. LPS/IFNγ-stimulated iNOS expression was reduced in Ssb-1^{T/+} and Ssb-2^{T/+} BMDM after 16 h stimulation (Time 0) and rapidly decreased post-wash in comparison to wild-type controls (FIGS. 9A and 10A), indicating that iNOS degradation was increased in these macrophages. Furthermore, the enhanced iNOS degradation was not seen in BMDM from mice that express either the Ssb-1 or Ssb-2 transgene lacking the SOCS box (Ssb-1ΔSB^{T/+}, Ssb-2ΔSB^{T/+}), indicating that the SSB regulation of iNOS is SOCS box-dependent (FIGS. 9B and 10B). Macrophage expression of the SSB transgenes was confirmed by anti-FLAG immunoprecipitation and Western blot (FIGS. 9C and 10C, top panels). Re-blot of the anti-FLAG immunoprecipitates with anti-iNOS antibody showed co-immunoprecipitation of iNOS with both full-length and SOCS box-deleted forms of SSB-1 and SSB-2. Notably, more iNOS appeared to co-immunoprecipitate with SSB-2 than SSB-1 (FIGS. 9C and 10C).

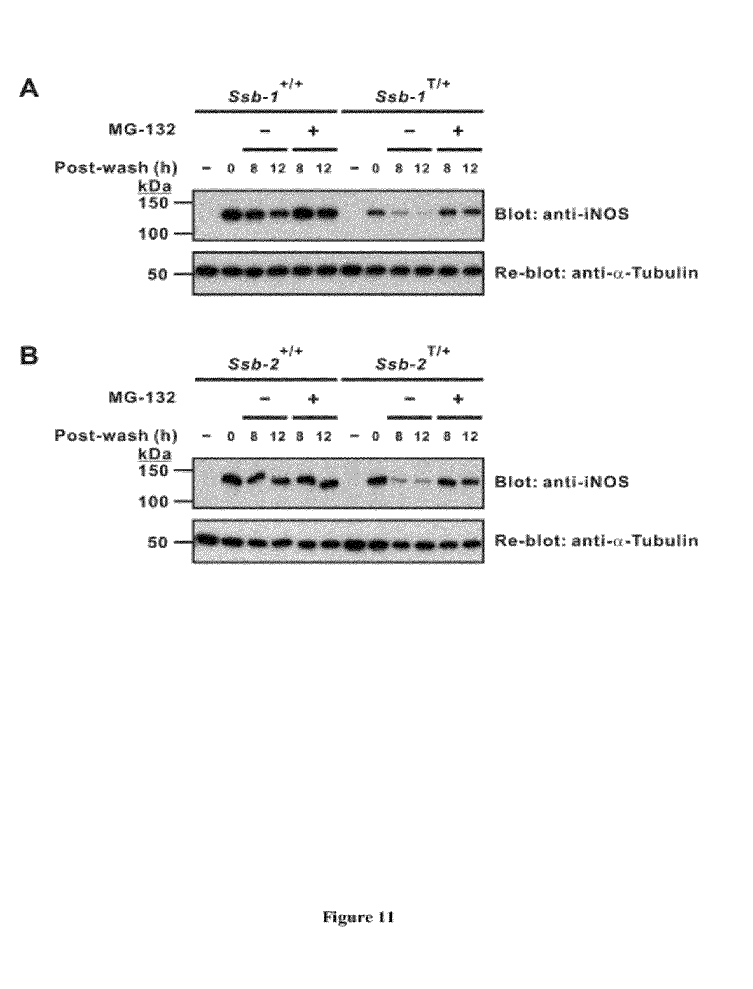

The regulation of iNOS protein by SSB-1 and SSB-2 is dependent on the proteasome, as the enhanced iNOS degradation was abrogated in Ssb-1^{T/+} and Ssb-2^{T/+} BMDM treated with the proteasomal inhibitor MG-132 post-wash (FIGS. 11A and 11B).

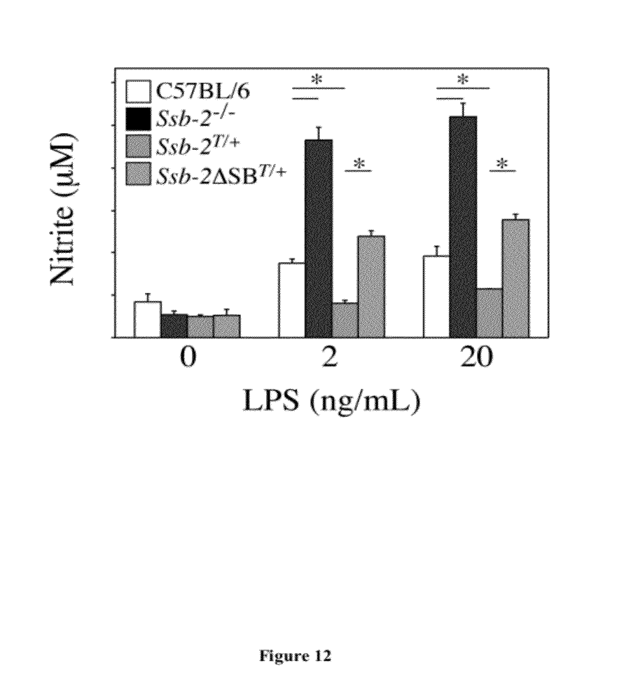

To determine whether the change in iNOS expression observed in Ssb-2^{-/-} and Ssb-2^{T/+} macrophages translated to a change in production of nitric oxide, BMDM from C57BL/6, Ssb-2^{-/-}, and Ssb-2ΔSB^{T/+} mice were cultured for 24 hours with 2 or 20 ng/ml LPS and culture supernatant assayed for production of nitrite using the Griess reagent. Macrophages from Ssb2^{-/-} mice produced significantly more nitrite compared to C57BL/6 macrophages, whilst macrophages from Ssb-2^{T/+} mice produced significantly less nitrate compared to wild-type controls. The suppression of nitrite production by SSB-2 was shown to be SOCS box-dependent, as macrophages expressing the SOCS box-deleted transgene (Ssb-2ΔSB^{T/+}) produced nitrite at comparable levels to wild-type controls (FIG. 12).

### Example 6

### iNOS Peptide can Competitively Inhibit the iNOS/SSB2 Interaction and in Vitro Ubiquitination of iNOS

### Materials and Methods

### Ubiquitin Cascade Components

Human E1 (GST tagged) was purchased from Biomol International (U.S.A). Bovine ubiquitin was purchased from Sigma-Aldrich.

### Cloning and Expression of the Cullin5/Rbx2 E3 Ligase Complex

Mouse Cullin5 was co-expressed as two domains, the N-terminal domain (1-384) and C-terminal domain (385-780). The C-terminal domain of Cullin5 was cloned into the second MCS of pACYCDUET (Novagen) whilst mouse Rbx2 was cloned into the first MCS resulting in a HIS6 tag at its N-terminus. The N-terminal domain of Cullin5 was cloned as a GST-fusion protein into pGEX-4T1 and the two vectors were co-expressed in BL21(DE3) cells to yield a ternary GST-Cul5(NTD)/HIS6-Rbx2/Cul(CTD) complex. Expression was performed in LB media at 18° C. overnight following induction using 1 mM IPTG when the OD₆₀₀ was 0.7. The cells were harvested by centrifugation and then lysed using lysozyme and sonication. The complex was bound to Ni-NTA resin, washed and eluted in buffer containing 250 mM imidazole. The eluant was then bound to Glutathione Sepharose (Amersham) and washed thoroughly in PBS to remove excess Rbx2. The complex was then eluted from the resin by thrombin proteolysis of the GST fusion tag and purified by size exclusion chromatography using a Superdex 200 16/60 column (Amersham). Finally, the purified E3 ligase was concentrated to 2 mg/mL.

### Cloning and Expression of Murine UbCH₅a

Murine UbCH5a (E2) was expressed as a GST-fusion protein by cloning into pGEX-4T and expressed in BL21(DE3) cells at 37° C. for two hours post IPTG induction. Following cell lysis, the protein was purified using Glutahione Sepharose and eluted by thrombin digestion. Size exclusion chromatography using a Superdex 75 16/60 column was performed as the final step in purification.

### Ubiquitination Assays

Ubiquitination assays were performed in 20 μl in 20 mM Tris-HCl, 50 mM NaCl, 5 mM MgCl2, 2.5 mM ATP, 0.1 mM DTT. Reactions were stopped by the addition of 2×SDS PAGE loading buffer and heating at 95° C. for 5 min. Reactions contained 0.1 μM E1, 2.5 μM E2, 2.5 μM E3, 50 μM ubiquitin and 5 μM SSB-2/elonginBC and were incubated for 30 minutes or as indicated at 37° C. Cell lysate was added as substrate. Results were visualised by Western Blot using anti iNOS monoclonal antibody following SDS-PAGE.

### Peptide Competition Assay

293T cells were transfected with vector alone or a construct expressing FLAG-tagged SSB-2 and lysed as described above. iNOS peptide (SEQ ID NO:3) was added in increasing concentrations (0.001, 0.01, 0.1, 1.0 and 10 μM) to iNOS-expressing lysate generated from C57BL/6 BMDM stimulated with LPS/IFNγ. 293T lysates were then added to the macrophage/iNOS peptide lysates and incubated for 1 h at 4° C. Anti-Flag antibody coupled to Sepharose beads (M2, Sigma) was added to the lysate mix and incubated for 3 h at 4° C. Complexes were then separated by SDS-PAGE and analysed by Western blot with anti-iNOS antibodies.

### Results

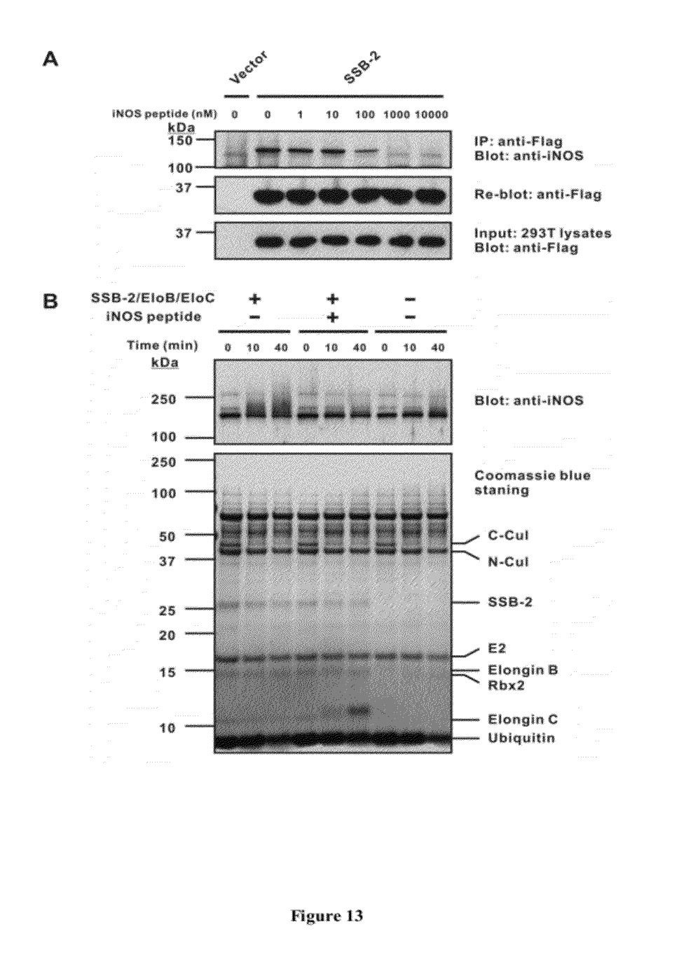

Given that there are a number of disease states where elevated or prolonged iNOS expression might be beneficial, the present inventors were interested in whether the iNOS/SSB-2 interaction could be disrupted. As a simple proof-of-concept experiment, free iNOS peptide was used to competitively disrupt the interaction. 293T cells were transiently transfected with cDNA expressing Flag-tagged SSB-2, lysed and mixed with iNOS-expressing macrophage lysates which contained increasing amounts of free iNOS peptide. The SSB-2 interaction with iNOS was modestly inhibited by 100 nM iNOS peptide and completely inhibited by 1000 nM peptide, as assessed by anti-Flag immunoprecipitation and Western blot with anti-iNOS antibodies (FIG. 13A).

SOCS box proteins recruit an E3 ubiquitin ligase complex which in the presence of E1 and E3 enzymes polyubiquitinates interacting proteins, targeting them for proteasomal degradation. A cell-free ubiquitination assay was established to demonstrate SSB-2 ubiquitination of iNOS. LPS/IFNγ stimulated macrophage lysates were used as a source of iNOS and incubated with ubiquitin and a trimeric SSB-2/elongin BC complex, in the presence of E1 and E2 enzymes, Rbx 1 and Cullin5. The reaction mixtures were then analysed by SDS-PAGE and Western blot with anti-iNOS antibodies. Enhanced. polyubiquitination of iNOS, as evidenced by higher molecular weight smearing, was observed after 10 and 40 min incubation in the presence of the SSB2/elongin BC complex. This was completely inhibited by the addition of free iNOS peptide (FIG. 13B, upper panel). Coomassie blue staining was used to demonstrate the relative levels of the E1, E2 and E3 components (FIG. 13B, lower panel).

### Example 7

### Increased Levels of iNOS Result in Enhanced Nitric Oxide in Ssb2^{-/-} Peritoneal Macrophages

### Materials and Methods

### Thioglycollate-Elicted Macrophages

Mice were injected intraperitoneally with 1 ml of aged 3% Brewer thioglycollate medium (Difco), sacrificed after 3 days and peritoneal cells harvested by lavage of the peritoneal cavity with PBS. Cells were cultured or for 24 hours in DMEM supplemented with 100 U/ml penicillin, 0.1 mg/ml streptomycin, 10% FBS and increasing concentrations of LPS or 20 ng/ml IFNγ/LPS. Aliquots of culture supernatant were retained for detection of nitric oxide using the Griess assay. Cultures were washed to remove LPS and non-adherent cells lysed for detection of iNOS by Western blot. FACS analysis confirmed that adherent cells were >98% +ve for CD11b (Mac-1).

### Results

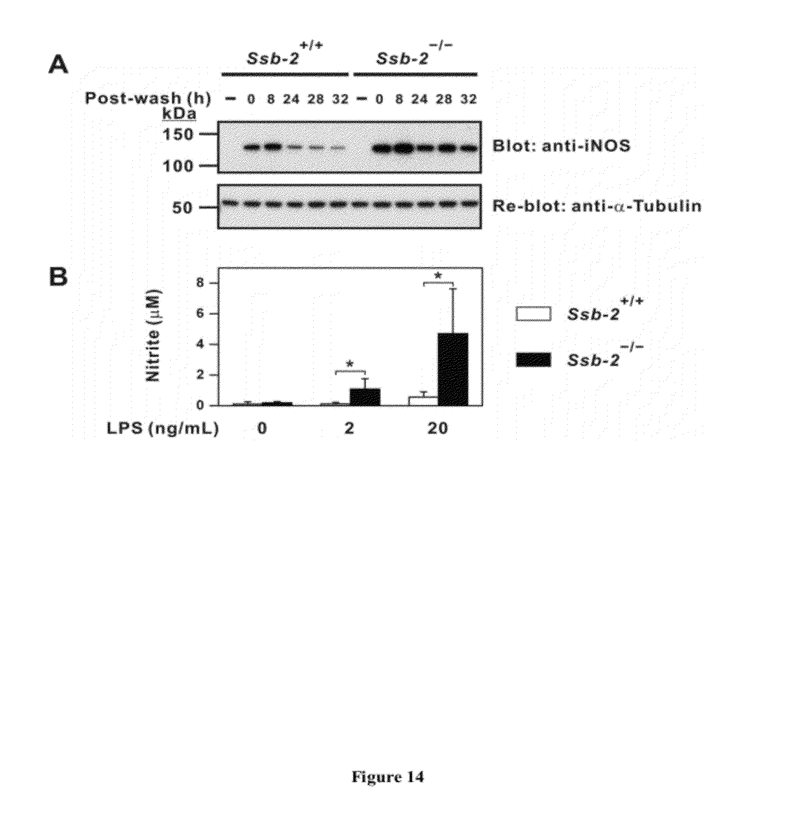

In addition to the data generated using macrophages derived in vitro, the inventors were interested in confirming the results in ex vivo macrophages. Peritoneal macrophages were elicited from Ssb-2^{-/-} mice and littermate controls by thioglycollate injection, cultured overnight with IFNγ and LPS, washed and harvested as described previously. iNOS expression post-wash was again prolonged in the SSB-2-deficient cells (FIG. 14A). To determine whether the change in iNOS expression translated to a change in production of nitric oxide, peritoneal macrophages were cultured for 24 hours with 2 or 20 ng/ml LPS and culture supernatant assayed for production of nitric oxide using the Griess reagent. Macrophages from Ssb2^{-/-} mice produced significantly more nitric oxide (FIG. 14B).

### Example 8

### SSB-2 Deficient Mice Show Enhanced Killing of Leishmania major

### Materials and Methods

Bone-marrow derived macrophages were plated onto glass coverslips at a density of 5×10⁴ macrophages per well in 0.5 ml DME with 10% foetal bovine serum and allowed to adhere for 3 days at 37° C. in a humidified atmosphere with 10% CO₂. Nonadherent cells were washed, and infected with L. major promastigotes at a ratio of 10:1 for 4 h. Cells were then washed and incubated for up to 48 h, fixed and stained with Giemsa (Scott et al., 2000).

### Results

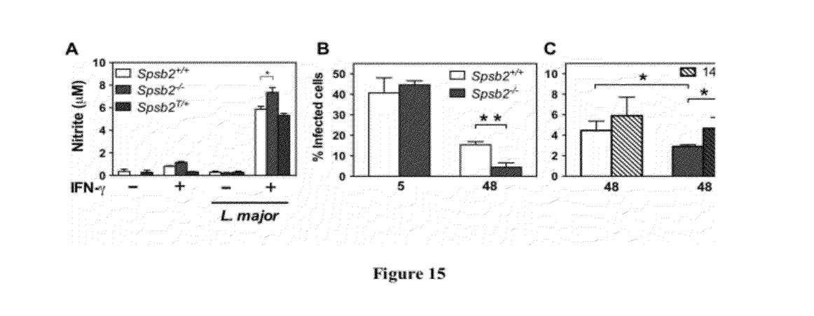

The elevated NO production by SSB-2-deficient macrophages correlates with increased parasite killing in vitro (FIG. 15). Macrophages from SSB-2-deficient mice (Ssb-2^{-/-}) showed enhanced killing of Leishmania parasites at 24 and 48 h when compared with macrophages derived from C57BL/6 mice, and this effect was enhanced in the presence of TNFα.

### Example 9

### shRNA Mediated Knockdown of SPSB1

### Materials and Methods

Oligonucleotides targeting SPSB 1 (CCAGATGCAGAGAATAAACTA (SEQ ID NO:84)) were designed as previously described (27). shRNAmir constructs were created by annealing the oligonucleotides in 5× annealing buffer (0.5 M potassium acetate, 0.01 M magnesium acetate and 0.15 M HEPES pH 7.4) for 5 min at 95° C., followed by incubation for 10 min at 80° C. and a 5-7 h ramp from 80° C. to 4° C. (reducing by 0.5° C. every 2.5 min). Annealed oligonucleotides were subsequently subcloned into the LMP vector (Dickins et al; 2005 & 2006). Non-sense shRNAmir and luciferase control constructs in the LMS vector were a kind gift from Dr. Marnie Blewitt (Majewski et al, 2008) and Dr Ross Dickins (unpublished data) respectively. To create retrovirus, 293T cells were transfected as described previously (Majewski et al, 2008). The medium was replaced with DMEM containing 10% FBS and 20% L-cell conditioned medium 24 h after transfection and viral supernatants harvested the following day. Total bone marrow was collected and non-adherent haematopoietic cells were harvested by centrifugation and red blood cells removed by washing in red cell removal buffer (154.4 mM NH₄Cl, 0.1 mM EDTA, 12 mM. NaHCO₃). Retroviral supernatants were applied to culture dishes pre-treated with 32 μg/ml RetroNectin (Takara Biosciences, Shiga, Japan) and centrifuged for 1 h at 4000 g at 4° C. Bone marrow cells were infected by co-culturing with the virus in the presence of 4 μg/ml 30 polybrene-containing medium for 24 h. Cells were removed from dish and fresh DMEM containing 10% FBS and 20% L-cell conditioned medium added and incubated for 48 h after which 2 μg/ml puromycin was added to select for infected cells. 6 days post-infection adherent macrophages were harvested and plated for subsequent experiments.

### Results

shRNA knockdown of Spsb1 leads to Earlier Induction of iNOS

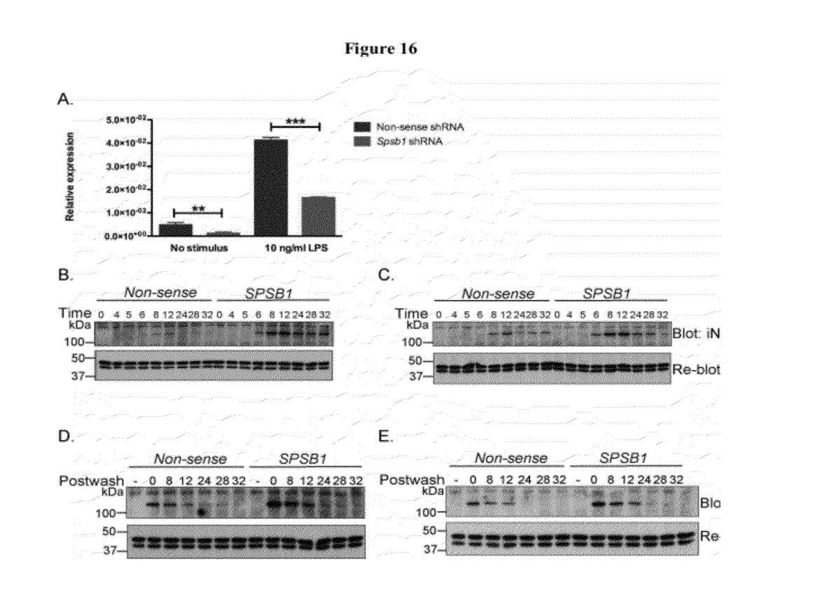

Over-expression of Spsb1 led to a reduction of iNOS in response to LPS and PolyIC and enhanced the degradation of iNOS. To determine the physiological relevance of the SPSB1/iNOS interaction, we employed short hairpin (sh) RNA technology to reduce Spsb1 expression in BMDM. Retroviral shRNA constructs were designed to target Spsb1 or a nonspecific sequence (Non-sense). To confirm the shRNA construct was able to effectively knockdown Spsb1 expression, BMDM, infected with either a non-sense control shRNA or Spsb1 shRNA were incubated with or without 10 ng/ml LPS for 4 h followed by Q-PCR analysis for Spsb1 mRNA levels. Spsb1 expression was significantly reduced (p<0.0001) in BMDM infected with Spsb1 shRNA, compared to BMDM infected with non-sense control shRNA (FIG. 16A).

To analyse the kinetics of iNOS induction in BMDM infected with an Spsb1 shRNA construct cells were stimulated with 100 ng/ml LPS or 25 μg/ml PolyIC for various times, then lysed and iNOS expression analysed by Western blot. BMDM infected with non-sense shRNA showed an induction of iNOS beginning at 8 h post-treatment, which continued throughout the timecourse (FIG. 16B). BMDM infected with Spsb1 shRNA, however, displayed a change in the kinetics with expression of iNOS observed earlier at 6 h post-treatment (FIG. 16C). In addition, there appears to be more iNOS protein present in BMDM infected with Spsb1 shRNA, presumably due to the earlier induction of iNOS and subsequent accumulation. In a degradation experiment where BMDM were incubated in 20 ng/ml LPS or 25 μg/ml. PolyIC overnight and the stimulus removed, an increased amount of iNOS was observed in BMDM infected with Spsb1 shRNA compared to non-sense infected BMDM. However, the kinetics of iNOS clearance remained essentially unchanged (FIGS. 16D and E).

Knockdown of Spsb1 expression Leads to increased Levels of Nitric Oxide

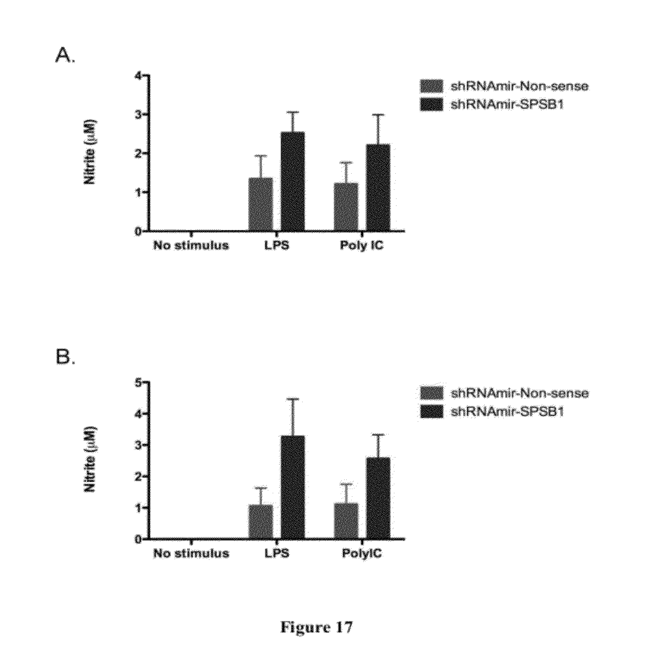

Using a Griess assay, NO production was measured in response to 20 ng/ml LPS and 25 μg/ml PolyIC in BMDM infected with non-sense or Spsb1 shRNA at 24 h and 48 h. At 24 h there was an increase in NO production in Spsb1 shRNA infected BMDM compared to non-sense infected BMDM in response to LPS and PolyIC (FIG. 17A). At 48 h this continued with a significant increase (p=0.0446) in NO production in Spsb1 shRNA infected BMDM in response to LPS and an increase in response to PolyIC (FIG. 17B).

### Example 10

### SPSB2 Regulation of iNOS Results in Altered Nitric Oxide Output

### Materials and Methods

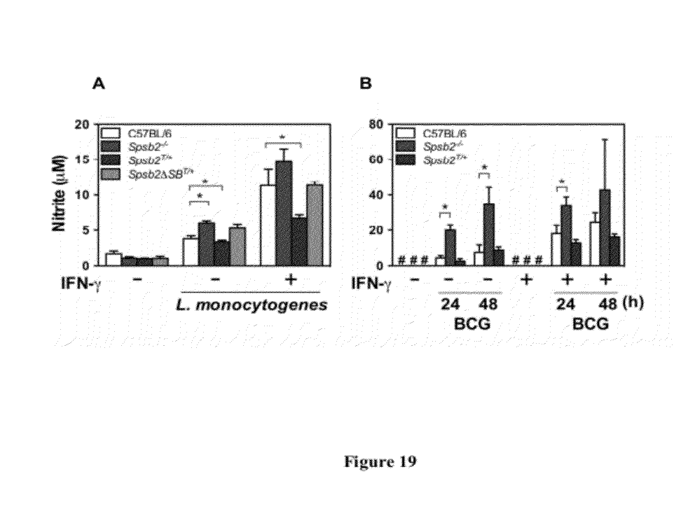

To determine whether SPSB2 regulates NO production in response to live bacilli, BMDM from C57BL/6, Spsb2^{-/-}, Spsb2^{T/+} and Spsb2ΔSB^{T/+} mice were pre-incubated with or without IFN-γ, washed, and infected with Listeria monocytogenes. Cells were then washed and cultured in DMEM containing 10 μg/ml gentamicin, a membrane-impermeant antibiotic, and NO₂⁻ production was measured 16 h post-infection. Spsb2^{-/-} BMDM produced slightly more NO₂⁻ than wild-type macrophages, while NO₂⁻ generation by Spsb2^{T/+} BMDM was comparable to wild-type in the absence of IFN-γ and reduced in the presence of IFN-γ (FIG. 19A). Compared to that induced by LPS, the difference in NO production between Spsb2^{-/-} and wild-type macrophages appeared to be modest in response to Listeria infection. In comparison, Spsb2^{-/-} BMDM infected with M. bovis (BCG) produced more NO₂⁻ than wild-type BMDM 24 and 48 h post-infection; NO₂⁻ production was augmented in the presence of IFN-γ, and by 48 h the amounts were similar between wild-type and Spsb2^{-/-} cells (FIG. 19B).

### Results

Enhanced nitric oxide levels were observed in SPSB2-deficient cells following challenge with gram-positive Listeria and mycobacteria, and also with Leishmania parasites (Example 8) and endotoxin (LPS; Examples 5 & 7), all of which trigger host responses via different Toll-like receptors and signaling pathways to converge on the rapid induction of iNOS.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

The present application claims priority from U.S. 61/176,637 filed 8 May 2009, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Almeida and Allshire (2005) Trends Cell Biol, 15:251-258.
Bird et al. (1988) Science, 242:423-426.
Constantini et al. (2008) Cancer Biotherm Radiopharm, 23: 3-24.
de Castro et al. (2006) Nucleic Acids Res, 34 (Web Server Issue):W362.
De Coupade et al. (2005) Biochem J, 390:407-418.
Deshayes et al. (2008) Adv Drug Deliv Rev, 60:537-47.
Dickins et al. (2005) Nat. Genet. 37:1289-1295
Dickins et al (2006) Nat. Genet. 39:914-921
Dosztanyi et al. (2005) Bioinformatics, 21:3433-3434.
DuBridge et al. (1987) Mol Cell Biol, 7:379-387.
Gallop et al. (1994) J Med Chem, 37:1233-1251.
Harmsen and De Haard (2007) Appl Microbiol Biotechnol, 77:13-22.
Hilton et al. (1998) Proc Natl Acad Sci USA, 95:114-119.
Horton et al. (1989) Gene, 77:61-68.
Howl et al. (2007) Biochem Soc Trans 35:767-769.
Huston et al. (1988) Proc Natl Acad. Sci. USA. 85:5879-5883.
Jones et al. (1986) Nature. 321:522-525.
Koppelhus et al. (2008) Bioconj Chem, 19:1526-34.
Look et al. (1996) Bioorg and Med Chem Letters, 6:707-712.
Majewski et al. (2008) PLoS Biol. 6:e93
Masters et al. (2005) Mol Cell Biol, 25:5639-5647.
Meyer-Losic et al. (2006) J Med Chem, 49:6908-6916.
Millar and Waterhouse (2005) Funct Integr Genomics, 5:129-135.
Mizushima and Nagata (1990) Nucleic Acids Res, 18:5322.
Morrison et al. (1984) Proc Natl Acad Sci USA, 81:6851-6855.
Muyldermans (2001) J Biotechnol 74:277-302.
Nicholoson et al. (1995) Blood, 86:3698-3704.
Pasquinelli et al. (2005) Curr Opin Genet Develop, 15:200-205.
Perriman et al. (1992) Gene, 113:157-163.
Prilusky et al. (2005) Bioinformatics, 21:3435-3438.
Ruhland et al. (1996) J Amer Chem Soc, 111:253-254.
Scott et al. (2000) Microbes Infect, 2:1131-1138.
Shippy et al. (1999) Mol Biotech, 12:117-129.
Styhler et al. (2002) Dev Cell, 3:865-876.
Tibary et al. (2007) Soc Reprod Fertil Suppl 64:297-313.
Visintin et al. (2008a) J Biotechnol, 135:1-15.
Visintin et al. (2008b) J Immunol Methods, 290:135-53.
Wang et al. (2005) J Biol Chem, 280:16393-16401.
Wishart et al. (1995) J Biomol NMR, 6:135-140.
Woo et al. (2006) Mol Cell, 24:967-976.
Wormald et al. (2006) J Biol Chem, 281:11135-11143.

1. A method of modulating the level of inducible nitric oxide synthetase (iNOS) in a cell, the method comprising administering to the cell a compound which modulates binding of SPRY domain-containing SOCS box protein (SSB) to iNOS, and/or a compound which modulates SSB activity in the cell.

2. The method of claim 1, wherein the method comprises administering to the cell a compound which inhibits binding of SSB to iNOS and/or a compound which reduces the level of SSB activity in the cell, whereby the level of iNOS in the cell is increased.

3. A method of treating or preventing a disease in a subject in accordance with the method of claim 1, the method comprising administering a compound which inhibits binding of SSB to iNOS in a cell of the subject and/or a compound which reduces the level of SSB activity in the cell.

4. The method of claim 3, wherein the disease is selected from tuberculosis, pneumonia, malaria, listeriosis, amebiasis, candidiasis, trichomoniasis, mycoplasmosis, paracoccidioidomycosis, leishmaniasis, bovine tuberculosis, Johne's disease, porcine enzootic pneumonia, or cancer.

5. The method of claim 3, wherein the disease is caused by infection with Mycobacterium, Samonella, Toxoplasmasa gondii, Helicobacter pylori, Chlamydia, Chlamydophila, Staphylococcus, Escerichia coli, Klebsiella, Pseudomonas, Streptococcus, Burkholderia, Leishmania, Plasmodium or Listeria.

6. The method of claim 5, wherein the Mycobacterium infection is infection with Mycobacterium tuberculosis, Mycobacterium leprae, Mycobacterium lepromatosis, Mycobacterium avium, Mycobacterium bovis, Mycobacterium avium sub. paratuberculosis or Mycobacterium ulcerans; the Plasmodium infection is infection with Plasmodium falciparum, Plasmodium vivax, Plasmodium ovale, Plasmodium malariae, or Plasmodium knowlesi; or the Leishmania infection is infection with Leishmania major, Leishmania mexicana, Leishmania tropica, Leishmania aethiopica, Leishmania braziliensis, Leishmania donovani, or Leishmania infantum.

7. The method of claim 1, wherein the compound binds to SSB and inhibits the binding of SSB to iNOS.

8. The method of claim 7, wherein the compound is a peptide comprising:
i) an amino acid sequence as provided in any one of SEQ ID NOs:1 to 22,
ii) an amino acid sequence which is at least 80% identical to any one of SEQ ID NOs:1 to 22, and/or
iii) a biologically active fragment of i) or ii).

9. The method of claim 8, wherein the peptide is 20 or fewer residues in length.

10. The method of claim 7, wherein the compound is a mimetic of the peptide defined in claim 8.

11. The method of claim 7, wherein the compound is an antibody that binds SSB.

12. The method of claim 11, wherein the antibody binds to amino acid residues within:
i) an amino acid sequence as provided in any one of SEQ ID NOs:64 to 82, and/or
ii) an amino acid sequence which is at least 80% identical to any one of SEQ ID NOs:64 to 82.

13. The method of claim 12, wherein the antibody binds to one or more of residues E55, N56, R68, P70, A72, R100, G101, T102, H103, Y120, L123, L124, L125, 5126, N127, 5128, V206, W207 or G208 of SEQ ID NO:64, or to an epitope which comprises one or more of said residues.

14. The method of claim 7, wherein the compound is functionally inactive iNOS, or an isolated polynucleotide encoding the functionally inactive iNOS.

15. The method of claim 1, wherein the compound binds to iNOS and inhibits the binding of iNOS to SSB.

16. The method of claim 15, wherein the compound is an isolated polypeptide comprising the SPRY domain of SSB, or an isolated polynucleotide encoding the polypeptide, wherein the polypeptide does not have SSB activity.

17. The method of claim 15, wherein the compound is an antibody which binds iNOS and inhibits binding of iNOS to SSB in a cell.

18. The method of claim 16, wherein the compound is an antibody that binds to amino acid residues within:
i) an amino acid sequence as provided in any one of SEQ ID NOs:1 to 22, and/or
ii) an amino acid sequence which is at least 80% identical to any one of SEQ ID NOs:1 to 22.

19. The method of claim 1, wherein the compound is an isolated polynucleotide which reduces the level of SSB activity in the cell and/or a construct encoding the polynucleotide.

20. The method of claim 19, wherein the polynucleotide is selected from: an antisense polynucleotide, a sense polynucleotide, a catalytic polynucleotide, a microRNA, and a double-stranded RNA.

21. The method of claim 20, wherein the polynucleotide is a siRNA or shRNA.

22. The method of claim 1, wherein the method comprises administering to the cell a compound which increases SSB activity in the cell, whereby the level of iNOS in the cell is reduced.

23. A method of treating or preventing a disease in a subject in accordance with the method of claim 1, the method comprising administering to the cell a compound which increases SSB activity in the cell, whereby the level of iNOS in the cell is reduced.

24. The method of claim 23, wherein the disease is sepsis-induced lung injury, asthma or shock, or is caused by excessive inflammation and/or excessive cytokine production.

25. The method of claim 24, wherein the cytokine is TNFα, IFNγ, IFNβ and/or IFNα.

26. The method of claim 22, wherein the compound is an isolated polypeptide comprising the SPRY domain and SOCS box of SSB, or a polynucleotide encoding the polypeptide, wherein the polypeptide has SSB activity.

27. The method of claim 1, wherein the SSB is SSB-1, 2 or 4.

28. An isolated peptide or mimetic thereof, wherein the peptide consists of:
i) an amino acid sequence as provided in any one of SEQ ID NOs:1 to 22,
ii) an amino acid sequence which is at least 80% identical to any one of SEQ ID NOs:1 to 22, and/or
iii) a biologically active fragment of i) or ii).

29. The peptide or mimetic thereof of claim 28, wherein the peptide is 20 or fewer residues in length.

30. An isolated antibody selected from an antibody which binds to SSB and inhibits binding of SSB to iNOS in a cell, and an antibody which binds iNOS and inhibits binding of iNOS to SSB in a cell.

31. The antibody of claim 30 which binds to SSB and inhibits binding of SSB to iNOS in a cell, wherein the antibody binds to amino acid residues within:
i) an amino acid sequence as provided in SEQ ID NO:64 to 82, and/or
ii) an amino acid sequence which is at least 80% identical to SEQ ID NO:64 to 82.

32. The antibody of claim 31, wherein the antibody binds to one or more of residues E55, N56, R68, P70, A72, R100, G101, T102, H103, Y120, L123, L124, L125, 5126, N127, 5128, V206, W207 or G208 of SEQ ID NO:64, or to an epitope which comprises one or more of said residues.

33. (canceled)

34. The antibody of claim 30 which binds iNOS and inhibits binding of iNOS to SSB in a cell, wherein the antibody binds to amino acid residues within:
i) an amino acid sequence as provided in any one of SEQ ID NOs:1 to 22, or
ii) an amino acid sequence which is at least 80% identical to any one of SEQ ID NOs:1 to 22.

35. The antibody of claim 30 which is fused and/or conjugated to a cell targeting agent or a cell penetrating agent.

36. (canceled)

37. (canceled)

38. A pharmaceutical composition comprising the peptide or mimetic thereof of claim 28.

39. (canceled)

40. A method for identifying an inhibitor of the binding of SSB to iNOS, the method comprises the steps of:
i) contacting SSB, or an iNOS binding fragment thereof, or iNOS, or a SSB binding fragment thereof, with one or more candidate compounds,
ii) identifying a candidate compound which binds to SSB or iNOS, and
iii) determining whether the candidate compound inhibits the binding of SSB to iNOS.

41. The method of claim 40, wherein the candidate compound which binds to SSB or iNOS is identified by surface plasmon resonance or high-resolution NMR.

42. The method of claim 40, wherein step iii) comprises:
a) incubating iNOS, or a SSB binding fragment thereof, with SSB, or an iNOS binding fragment thereof, with the candidate compound under conditions sufficient for SSB to bind to iNOS to form a complex, and
b) determining if the candidate compound inhibits the formation of the complex.

43. The method of claim 40, wherein the candidate compound is a peptide or mimetic thereof, or an antibody.

44. The peptide or mimetic thereof of claim 28 which is fused and/or conjugated to a cell targeting agent or a cell penetrating agent.

45. A pharmaceutical composition comprising the antibody of claim 30.

46. The pharmaceutical composition of claim 38, comprising said peptide or mimetic thereof, and an isolated antibody selected from an antibody which binds to SSB and inhibits binding of SSB to iNOS in a cell, and an antibody which binds iNOS and inhibits binding of iNOS to SSB in a cell.


SEQUENCE LISTING (84 sequences)

SEQ ID NO: 1 - protein, 60 aa, Homo sapiens
MACPWKFLFKTKFHQYAMNGEKDINNNVEKAPCATSSPVTQDDLQYHNLSKQQNESPQPL

SEQ ID NO: 2 - protein, 13 aa, Artificial Sequence
  peptide Homo sapiens iNOS motif
NGEKDINNNVEKA

SEQ ID NO: 3 - protein, 13 aa, Artificial Sequence
  peptide synthesized iNOS motif
KEEKDINNNVKKT

SEQ ID NO: 4 - protein, 13 aa, Artificial Sequence
  peptide synthesized iNOS motif
KEAKDINNNVKKT

SEQ ID NO: 5 - protein, 13 aa, Artificial Sequence
  peptide synthesized iNOS motif
KEEADINNNVKKT

SEQ ID NO: 6 - protein, 13 aa, Artificial Sequence
  peptide synthesized iNOS motif
KEAADINNNVKKT

SEQ ID NO: 7 - protein, 13 aa, Artificial Sequence
  peptide synthesized iNOS motif
KEEKAINNNVKKT

SEQ ID NO: 8 - protein, 13 aa, Artificial Sequence
  peptide synthesized iNOS motif
KEEKDANNNVKKT

SEQ ID NO: 9 - protein, 13 aa, Artificial Sequence
  peptide synthesized iNOS motif
KEEKDIANNVKKT

SEQ ID NO: 10 - protein, 13 aa, Artificial Sequence
  peptide synthesized iNOS motif
KEEKDIQNNVKKT

SEQ ID NO: 11 - protein, 13 aa, Artificial Sequence
  peptide synthesized iNOS motif
KEEKDINANVKKT

SEQ ID NO: 12 - protein, 13 aa, Artificial Sequence
  peptide synthesized iNOS motif
KEEKDINNAVKKT

SEQ ID NO: 13 - protein, 13 aa, Artificial Sequence
  peptide synthesized iNOS motif
KEEKDINNQVKKT

SEQ ID NO: 14 - protein, 13 aa, Artificial Sequence
  peptide synthesized iNOS motif
KEEKDINNNAKKT

SEQ ID NO: 15 - protein, 13 aa, Artificial Sequence
  peptide synthesized iNOS motif
KEEKDINNNVKAT

SEQ ID NO: 16 - protein, 13 aa, Artificial Sequence
  peptide synthesized iNOS motif
KEEKDINNNAKAT

SEQ ID NO: 17 - protein, 13 aa, Artificial Sequence
  peptide synthesized iNOS motif
KEEADINNNAKAT

SEQ ID NO: 18 - protein, 13 aa, Artificial Sequence
  peptide rat iNOS motif
KEEKDINNNVEKT

SEQ ID NO: 19 - protein, 13 aa, Artificial Sequence
  peptide bovine iNOS motif
ATELDINNNVGKF

SEQ ID NO: 20 - protein, 13 aa, Artificial Sequence
  peptide canine iNOS motif
KEEKDINNNVEKP

SEQ ID NO: 21 - protein, 13 aa, Artificial Sequence
  peptide guinea pig iNOS motif
TEEKDINNNVGKA

SEQ ID NO: 22 - protein, 13 aa, Artificial Sequence
  peptide chicken iNOS motif
SEEKDINNNVEKD

SEQ ID NO: 23 - DNA, 21 bp, Artificial Sequence
  oligonucleotide primer
ttgtcaagctcatttcctggt

SEQ ID NO: 24 - DNA, 21 bp, Artificial Sequence
  oligonucleotide primer
ttactccttggaggccatgta

SEQ ID NO: 25 - DNA, 19 bp, Artificial Sequence
  oligonucleotide primer
cggggactcaagggtaaaa

SEQ ID NO: 26 - DNA, 19 bp, Artificial Sequence
  oligonucleotide primer
aggggctcaggatcaagtc

SEQ ID NO: 27 - DNA, 23 bp, Artificial Sequence
  oligonucleotide primer
aagaagagtggaggaaccacaat

SEQ ID NO: 28 - DNA, 23 bp, Artificial Sequence
  oligonucleotide primer
caaaggcagagtggatatttgac

SEQ ID NO: 29 - DNA, 24 bp, Artificial Sequence
  oligonucleotide primer
gcagctctaactgggctatgactc

SEQ ID NO: 30 - DNA, 25 bp, Artificial Sequence
  oligonucleotide primer
acaggcacagcactggggatggatg

SEQ ID NO: 31 - DNA, 18 bp, Artificial Sequence
  oligonucleotide primer
gagtgctgtgtggggtca

SEQ ID NO: 32 - DNA, 18 bp, Artificial Sequence
  oligonucleotide primer
agggctgagcggatggat

SEQ ID NO: 33 - DNA, 20 bp, Artificial Sequence
  oligonucleotide primer
agatcgagccctggaagacc

SEQ ID NO: 34 - DNA, 23 bp, Artificial Sequence
  oligonucleotide primer
attagcatggaagcaaagaacac

SEQ ID NO: 35 - protein, 10 aa, Artificial Sequence
  motif
EKDINNNVXK

SEQ ID NO: 36 - protein, 6 aa, Artificial Sequence
  motif sequence
XXNNNX

SEQ ID NO: 37 - protein, 8 aa, Artificial Sequence
  Flag epitope
DYKDDDDK

SEQ ID NO: 38 - protein, 6 aa, Artificial Sequence
  vector N-terminus residues
GSSARQ

SEQ ID NO: 39 - protein, 7 aa, Artificial Sequence
  vector C-terminus residues
TRRIHRD

SEQ ID NO: 40 - DNA, 792 bp, Homo sapiens
atgggccagacagctctggcagggggcagcagcagcacccccacgccacaggccctgtaccctgacctctcctgtcccgagggcttggaagagctgctgtctgcaccccctcctgacctgggggcccagcggcgccacggttggaaccccaaagactgttcagagaacatcgaggtcaaggaaggagggttgtactttgagcggcggcccgtggcccagagcactgatggggcccggggtaagaggggctattcaaggggcctgcacgcctgggagatcagctggcccctagagcagaggggcacgcatgccgtggtgggcgtggccacggccctcgccccgctgcagactgaccactacgcggcgctgctgggcagcaacagcgagtcgtggggctgggacatcgggcgggggaagctgtaccatcagagcaaggggcccggagccccccagtatccagcgggaactcagggtgagcagctggaggtgccagagagactgctggtggttctggacatggaggagggaactctgggctacgctattgggggcacctacctggggccagcattccgcggactgaagggcaggaccctctatccggcagtaagcgctgtctggggccagtgccaggtccgcatccgctacctgggcgaaaggagagcggagccacactcccttctgcacctgagccgcctgtgtgtgcgccacaacctgggggatacccggctcggccaggtgtctgccctgcccttgccccctgccatgaagcgctacctgctctaccagtga

SEQ ID NO: 41 - protein, 263 aa, Homo sapiens
MGQTALAGGSSSTPTPQALYPDLSCPEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPAGTQGEQLEVPERLLVVLDMEEGTLGYAIGGTYLGPAFRGLKGRTLYPAVSAVWGQCQVRIRYLGERRAEPHSLLHLSRLCVRHNLGDTRLGQVSALPLPPAMKRYLLYQ

SEQ ID NO: 42 - DNA, 795 bp, Mus musculus
atgggccagacggccctggcaaggggcagcagcagcacccctacctcgcaggctctgtactcggacttctctcctcccgagggcttggaggagctcctgtctgctccccctcctgacctggttgcccaacggcaccacggctggaaccccaaggattgctccgagaacatcgatgtcaaggaagggggtctgtgctttgagcggcgccctgtggcccagagcactgatggagtccgggggaaacggggctattcgagaggtctgcacgcctgggagatcagctggcccctggagcaaaggggcacacacgccgtggtgggcgtggccaccgccctcgccccgctgcaggctgaccactatgcggcgcttttgggcagcaacagcgagtcctggggctgggatattgggcggggaaaattgtatcatcagagtaagggcctcgaggccccccagtatccagctggacctcagggtgagcagctagtggtgccagagagactgctggtggttctggacatggaggaggggactcttggctactctattgggggcacgtacctgggaccagccttccgtggactgaaggggaggaccctctatccctctgtaagtgctgtttggggccagtgccaggtccgcatccgctacatgggcgaaagaagagtggaggaaccacaatcccttctgcacctgagccgcctgtgtgtgcgccatgctctgggggacacccggctgggtcaaatatccactctgcctttgccccctgccatgaagcgctatctgctctacaaatga

SEQ ID NO: 43 - protein, 264 aa, Mus musculus
MGQTALARGSSSTPTSQALYSDFSPPEGLEELLSAPPPDLVAQRHHGWNPKDCSENIDVKEGGLCFERRPVAQSTDGVRGKRGYSRGLHAWEISWPLEQRGTHAVVGVATALAPLQADHYAALLGSNSESWGWDIGRGKLYHQSKGLEAPQYPAGPQGEQLVVPERLLVVLDMEEGTLGYSIGGTYLGPAFRGLKGRTLYPSVSAVWGQCQVRIRYMGERRVEEPQSLLHLSRLCVRHALGDTRLGQISTLPLPPAMKRYLLYK

SEQ ID NO: 44 - DNA, 792 bp, Canis familiaris
atgggccagacggccctggcagggggcagcagcagcacccccaccccacagaccctgtaccctgacctctcttgtcctgagggcttggaggagctgttgtctgctccccctcctgatctgggagcccaacagcgccacggctggaaccccaaggactgctcagagaacatcgaggtcaaggaaggggggttatgctttgagcggcggccggtggcccagagcactgatggggcccggggtaagaagggctactcgaggggcctgcacgcctgggagatctgctggccccgggagcagaggggtacccacgccgtggtgggcgtggccacggccctcgccccgctgcaggctgaccactatgcggcgctgctgggcagcaacagcgagtcgtggggctgggacattgggcgagggaagctgtaccatcagagcaaggggcctggggcccctcagtatccagccagacctcacggtgagcagctggaagtgccggagaggctgctggtggttctggacatggaggagggaactctgggctacgctattgggggaacctacctggggccagccttccgcggactgaagggcaggaccctctatccggcagtaagcgctgtctggggccagtgccaggtccgcatccgctacctgggcggaaggagagcggagccacactcccttctgcacctgagccgcctgtgtgtgcgccacgccctgggggatacccggcttggccaggtatctgctctccccttgccccctgccatgaagcgctacctgctctaccagtga

SEQ ID NO: 45 - protein, 263 aa, Canis familiaris
MGQTALAGGSSSTPTPQTLYPDLSCPEGLEELLSAPPPDLGAQQRHGWNPKDCSENIEVKEGGLCFERRPVAQSTDGARGKKGYSRGLHAWEICWPREQRGTHAVVGVATALAPLQADHYAALLGSNSESWGWDIGRGKLYHQSKGPGAPQYPARPHGEQLEVPERLLVVLDMEEGTLGYAIGGTYLGPAFRGLKGRTLYPAVSAVWGQCQVRIRYLGGRRAEPHSLLHLSRLCVRHALGDTRLGQVSALPLPPAMKRYLLYQ

SEQ ID NO: 46 - DNA, 822 bp, Homo sapiens
atgggtcagaaggtcactggagggatcaagactgtggacatgagggaccccacgtacaggcccctgaagcaggagctccagggtctggattactgcaagcccacccggctggatctgctactggacatgccccctgtgtcctatgatgtccagctgctgcattcatggaacaacaacgaccgatcgctcaatgtctttgtgaaggaggacgacaagctcatctttcaccggcatccggtggcccagagcacggacgctatcaggggcaaagtcgggtatacccgtgggctgcacgtgtggcagatcacgtgggccatgagacagcggggcacacacgccgtggtgggggtggcgacggcagacgcccccctgcactctgtcgggtacacaaccctcgtggggaataaccacgagtcctggggctgggacttggggcgcaaccggctctaccacgatggcaagaaccagccaagcaaaacatacccagcctttctggaaccagatgagacattcattgtccctgactccttcctggtagccctggacatggacgacgggactctgagcttcattgtggatggacagtacatgggagtggcttttcggggactcaagggcaaaaaactgtatcctgtagtgagtgccgtctggggccactgtgagatccgaatgcgctacttgaacggactcgatcccgagccgctgccgctcatggatttgtgccgtcgctcggtgcgcctggccctggggagggagcgcctgggggagatccacacgctgccgctgccggcttccctcaaggcctacctcctctaccagtga

SEQ ID NO: 47 - protein, 273 aa, Homo sapiens
MGQKVTGGIKTVDMRDPTYRPLKQELQGLDYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNGLDPEPLPLMDLCRRSVRLALGRERLGEIHTLPLPASLKAYLLYQ

SEQ ID NO: 48 - DNA, 822 bp, Mus musculus
atgggtcagaaggtcacaggagggatcaagactgtggacatgcgggaccccacataccgacctctgaagcaggaactccaggggctggattactgcaagcccacccggctggacctgctgctcgacatgccccctgtgtcctacgatgtgcagctgctccactcctggaacaataacgaccgttcgctcaacgtcttcgtgaaggaagatgacaagttgatctttcaccggcatccggtggcccagagcacggacgccatcaggggcaaagttgggtacacacgtggactgcatgtatggcagatcacatgggccatgaggcaacgaggcacgcatgccgtggtgggggtggccacagcagatgcccctttgcactccgttgggtacacaacccttgtaggaaataaccatgaatcctggggctgggacctggggcgtaaccgtctctaccacgacggcaagaaccagccaagtaaaacatacccagcctttctggagccggacgagacattcattgtccctgactcctttctcgtggccctggacatggatgatgggaccttaagtttcatcgtggatggacagtacatgggagtggctttccggggactcaagggtaaaaagctgtatcctgtagtgagtgccgtctggggccactgtgagatccgcatgcgctacttgaacggacttgatcctgagcccctgccactcatggacctgtgccggcgttcggtgcgcctagcgctgggaaaagagcgcctgggtgccatccccgctctgccgctacctgcctccctcaaagcctacctcctctaccagtga

SEQ ID NO: 49 - protein, 273 aa, Mus musculus
MGQKVTGGIKTVDMRDPTYRPLKQELQGLDYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNGLDPEPLPLMDLCRRSVRLALGKERLGAIPALPLPASLKAYLLYQ

SEQ ID NO: 50 - DNA, 1308 bp, Canis familiaris
atggccttgcatggcaaggaggtggccaggtatggacagcaggaggtctacgagaagaaagtgtcggctctgactgtagcgtggatgcagggtgggaagaggcaggtgacagacaaggggacagcccaggggccacgggaagagtatcactggtacccctggcccaggagggtcacagagctggctggggagttccctgtggcttcagggaaccgcaagccaggagaccccgaggccttccagctgagtgccatgcaagccctcaaatggacagcctgtcaccttcctggtggtgtggctgtggcgacagagccatggctgcggcctgccttgcatcaggcaccacggagggccctacagtccccagaggccgcctaccaaccttccttaggactgatctgggcttcattggaaaaccagacggaaggccccacgattgatgagtttgctttgggactcagtgcagaggtgaaaccaaaggcaaacatgggtcagaaggtcactggagggatcaagaccgtggacatgagggaccccacgtaccggcccctgaagcaggagctccagggcctggactactgcaagcccacccgcctggacctgctgctagacatgccccccgtgtcctacgacatccagctgctccactcatggaataacaatgaccgatcgctcaatgtctttgtgaaggaggacgacaagctaatctttcaccggcatccggtggcccagagtacagatgccatcaggggcaaagttgggtacacgcgtgggctgcatgtttggcagatcacgtgggccatgaggcagcggggcacacacgctgtggttggagtggcgacagcggatgcccccctgcactctgtcgggtatacaaccctcgtggggaacaaccatgagtcttggggctgggacttggggcgcaaccggctctatcatgatggcaaaaaccaaccaagcaaaacatacccggcctttttggagccggatgagacatttattgtccccgactccttcctcgtggccctggacatggatgacgggactctgagcttcattgtggacggacagtacatgggagtggcttttcgaggactcaagggcaaaaaactatatcctgtagtgagcgccgtctggggccactgtgagatccgcatgcgctacttgaatggacttgaccccgagcccctgccgctcatggatctctgccgccgctcagtgcgcctggccctcgggaaggagcgtctgggtgacatccacacgctgccgctgccggcctctctcaaggcctacctcctctaccagtga

SEQ ID NO: 51 - protein, 435 aa, Canis familiaris
MALHGKEVARYGQQEVYEKKVSALTVAWMQGGKRQVTDKGTAQGPREEYHWYPWPRRVTELAGEFPVASGNRKPGDPEAFQLSAMQALKWTACHLPGGVAVATEPWLRPALHQAPRRALQSPEAAYQPSLGLIWASLENQTEGPTIDEFALGLSAEVKPKANMGQKVTGGIKTVDMRDPTYRPLKQELQGLDYCKPTRLDLLLDMPPVSYDIQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNGLDPEPLPLMDLCRRSVRLALGKERLGDIHTLPLPASLKAYLLYQ

SEQ ID NO: 52 - DNA, 822 bp, Homo sapiens
atgggccagaagctctcggggagcctcaagtcagtggaggtgcgagagccggcgctgcggccggccaagcgggagctgcggggtgcagagcccgggcggccggcgcggctggaccagctgttggacatgccagcggcggggctggctgtgcagctgcggcacgcgtggaaccccgaggaccgctcgctcaacgtcttcgtcaaggacgacgaccggctcaccttccaccggcaccccgtggcccagagcaccgacggcatccgcggcaaggtgggccacgcccgcggcctgcacgcctggcagatcaactggccggctcggcagcgcggcacccacgctgtagttggtgtggccacggcccgtgctcccctgcactccgtgggctacacggcgctggtaggcagtgacgccgagtcgtggggctgggacctgggccgcagccgcctctaccacgacggcaagaaccagcccggcgtggcctacccggcctttctggggcccgacgaggcctttgcgctgcccgactcgctgctcgtggtgctggacatggatgagggcacactcagcttcatcgtggatggccagtacctgggcgtggccttccgaggtctcaagggcaagaagctgtacccggtggtgagtgccgtgtggggccactgtgaagtcaccatgcgctacatcaacggccttgaccccgagcccctgccactgatggacctgtgccggagatccatccgctcggccctgggccgccagcgcctgcaggacatcagctccctgcccctgcctcagtctctcaaaaactatctgcagtaccagtga

SEQ ID NO: 53 - protein, 273 aa, Homo sapiens
MGQKLSGSLKSVEVREPALRPAKRELRGAEPGRPARLDQLLDMPAAGLAVQLRHAWNPEDRSLNVFVKDDDRLTFHRHPVAQSTDGIRGKVGHARGLHAWQINWPARQRGTHAVVGVATARAPLHSVGYTALVGSDAESWGWDLGRSRLYHDGKNQPGVAYPAFLGPDEAFALPDSLLVVLDMDEGTLSFIVDGQYLGVAFRGLKGKKLYPVVSAVWGHCEVTMRYINGLDPEPLPLMDLCRRSIRSALGRQRLQDISSLPLPQSLKNYLQYQ

SEQ ID NO: 54 - DNA, 822 bp, Mus musculus
atgggtcagaagctctctgggagcctcaagtctgtggaggtgcgagagccagcgcttcggccagctaagcgggagctccgcggcttagaacctgggcgaccggcacgtctagaccagctgctggacatgcctgcggcagggttggcagtacagctgagacacgcgtggaaccccgaggaccgctccctcaatgtctttgtcaaggatgacgaccgcctcaccttccaccggcacccagtagcccagagcacagatggcatccgtggcaaggtgggccatgcccggggtctgcacgcatggcagatccattggcccgctaggcagcggggcacccacgctgtggtgggcgtggccacggcccgagcccctttgcactctgtgggttacaccgcactggtgggcagtgactccgagtcgtggggctgggacctgggtcgcagccgcctctaccacgacggcaagaaccggcctggggtggcatacccagccttcctggggccagatgaagcctttgctctgccagattccttattggtcgtactggacatggacgagggcacgctcagcttcatcgtggatggccagtacctgggcgtggccttccgtggcctcaagggcaagaaactatacccggtggtgagtgctgtgtggggtcactgcgaagtcaccatgcgctacatcaacggccttgaccctgagcctctgccgctgatggacctgtgccggcgatccatccgctcagccctgggccgacagcgcctgcgtgacatcggctccctgcccctgcctcagtctctgaaaaactacctgcagtaccagtga

SEQ ID NO: 55 - protein, 273 aa, Mus musculus
MGQKLSGSLKSVEVREPALRPAKRELRGLEPGRPARLDQLLDMPAAGLAVQLRHAWNPEDRSLNVFVKDDDRLTFHRHPVAQSTDGIRGKVGHARGLHAWQIHWPARQRGTHAVVGVATARAPLHSVGYTALVGSDSESWGWDLGRSRLYHDGKNRPGVAYPAFLGPDEAFALPDSLLVVLDMDEGTLSFIVDGQYLGVAFRGLKGKKLYPVVSAVWGHCEVTMRYINGLDPEPLPLMDLCRRSIRSALGRQRLRDIGSLPLPQSLKNYLQYQ

SEQ ID NO: 56 - DNA, 822 bp, Canis familiaris
atgggccagaagctgtcagggggcctcaagtcggtggaggtgcgcgagccggcgctgcggccggccaagcgggagctgcaggggccggagccggggcggccggcgcggctggaccagctgttggacatgcccgccgcggggctggcggtgcagctgcggcacgcctggaaccccgaggaccgctcgctcaacgtcttcgtcaaggacgacgaccggctcaccttccaccggcacccggtggcccagagcacggatggcatccggggcaaggtgggccacgcgcgcggcctgcacgcctggcagatcaactggccggcccggcagcgtggcacccacgccgtagtcggcgtggccactgctcgggcacctctgcactcggtgggctacacggccctggtgggcagtgacgccgagtcgtggggctgggacctgggccgcagccgcctctaccacgatgggaagaacaggcccggagtggcctaccccgccttcctggggcccgaggaggccttcgcgctgccggactcgctgctcgtggtgctggacatggacgagggcacgctcagcttcgtcgtggatggccagtacctgggcgtggccttccgcggcctcaagggcaagaagctgtacccggtggtcagtgccgtgtggggccactgcgaagtcaccatgcgctacatcaacggccttgaccccgagcccctgccgctgatggacctgtgccggagatccatccgcttggccctgggccgccagcgcctacaggacatcggcgccctgcccctgcctcagtctctcaaaaactatctgcagtaccagtga

SEQ ID NO: 57 - protein, 273 aa, Canis familiaris
MGQKLSGGLKSVEVREPALRPAKRELQGPEPGRPARLDQLLDMPAAGLAVQLRHAWNPEDRSLNVFVKDDDRLTFHRHPVAQSTDGIRGKVGHARGLHAWQINWPARQRGTHAVVGVATARAPLHSVGYTALVGSDAESWGWDLGRSRLYHDGKNRPGVAYPAFLGPEEAFALPDSLLVVLDMDEGTLSFVVDGQYLGVAFRGLKGKKLYPVVSAVWGHCEVTMRYINGLDPEPLPLMDLCRRSIRLALGRQRLQDIGALPLPQSLKNYLQYQ

SEQ ID NO: 58 - protein, 1153 aa, Homo sapiens
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

SEQ ID NO: 59 - protein, 1144 aa, Mus musculus
MACPWKFLFKVKSYQSDLKEEKDINNNVKKTPCAVLSPTIQDDPKSHQNGSPQLLTGTAQNVPESLDKLHVTSTRPQYVRIKNWGSGEILHDTLHHKATSDFTCKSKSCLGSIMNPKSLTRGPRDKPTPLEELLPHAIEFINQYYGSFKEAKIEEHLARLEAVTKEIETTGTYQLTLDELIFATKMAWRNAPRCIGRIQWSNLQVFDARNCSTAQEMFQHICRHILYATNNGNIRSAITVFPQRSDGKHDFRLWNSQLIRYAGYQMPDGTIRGDAATLEFTQLCIDLGWKPRYGRFDVLPLVLQADGQDPEVFEIPPDLVLEVTMEHPKYEWFQELGLKWYALPAVANMLLEVGGLEFPACPFNGWYMGTEIGVRDFCDTQRYNILEEVGRRMGLETHTLASLWKDRAVTEINVAVLHSFQKQNVTIMDHHTASESFMKHMQNEYRARGGCPADWIWLVPPVSGSITPVFHQEMLNYVLSPFYYYQIEPWKTHIWQNEKLRPRRREIRFRVLVKVVFFASMLMRKVMASRVRATVLFATETGKSEALARDLATLFSYAFNTKVVCMDQYKASTLEEEQLLLVVTSTFGNGDCPSNGQTLKKSLFMLRELNHTFRYAVFGLGSSMYPQFCAFAHDIDQKLSHLGASQLAPTGEGDELSGQEDAFRSWAVQTFRAACETFDVRSKHHIQIPKRFTSNATWEPQQYRLIQSPEPLDLNRALSSIHAKNVFTMRLKSQQNLQSEKSSRTTLLVQLTFEGSRGPSYLPGEHLGIFPGNQTALVQGILERVVDCPTPHQTVCLEVLDESGSYWVKDKRLPPCSLSQALTYFLDITTPPTQLQLHKLARFATDETDRQRLEALCQPSEYNDWKFSNNPTFLEVLEEFPSLHVPAAFLLSQLPILKPRYYSISSSQDHTPSEVHLTVAVVTYRTRDGQGPLHHGVCSTWIRNLKPQDPVPCFVRSVSGFQLPEDPSQPCILIGPGTGIAPFRSFWQQRLHDSQHKGLKGGRMSLVFGCRHPEEDHLYQEEMQEMVRKRVLFQVHTGYSRLPGKPKVYVQDILQKQLANEVLSVLHGEQGHLYICGDVRMARDVATTLKKLVATKLNLSEEQVEDYFFQLKSQKRYHEDIFGAVFSYGAKKGSALEEPKATRL

SEQ ID NO: 60 - protein, 1154 aa, Canis familiaris
MACPWKFLFRAKFHQYGMKEEKDINNNVEKPPGATPSPSTQDDLKNHKHHNDSPQPLTETVQKLPESLDKLHATPLSRPQHVRIKNWGNGRSFQDTLHHKAMGVLACTSKLCMGSIMNTKSLTRGPSDKPTPTEELLPQAIEFVNQYYGSFKEAKIEEHLARVEAVTKDIETTGTYQLTGDELIFATKQAWRNAPRCIGRIQWSNLQVFDARSCSTAKEMFEHICRHLRYASNNGNIRSAITVFPQRTDGKHDFRVWNAQLIRYAGYQMPDGTILGDPASVEFTQLCIDLGWKPKYGRFDVVPLVLQADGQDPEFFEIPPDLVLEVPMEHPKYEWFRELELKWYALPAVANMLLEVGGLEFPGCPFNGWYMGTEIGVRDFCDVQRYNILEEVGSKMGLETHKLASLWKDRAVIEINVAVLHSFQKQNVTIMDHHSAAESFMKYMQSEYRSRGGCPADWIWLVPPISGSITPVFHQEMLNYVLSPFYYYQVEAWKTHLWLDEKRRPHRKKIQLKVLVKAVLFASMLMRKTMASRVRVTILFATETGKSETLARDLGALFSCAFHPKVLCMDEYKLSHLEEEQLLLVVTSTFGNGDSPGNGEKLKKSLFMLKELTNNFRYAVFGLRSNMYPQFCAFAHDIDHKLSHLGASQLTPGGEGDELNGKEEAFRCWAVQTFKAACDTSDVRGKHCIQIPRLYTSNVTWDPHHYRLLQDSQPLDLNKALSKMHAKNVFTLRLKSQRNLQSPISNRTTLQVELSCEDSQELSYLPGEHLGVFPGNQLALVQGILERVVYSPAPLQPVHLETLSERGSYWVRNNRLPPCSLSQALTYFLDITTPPTHLLLRKLAQLAHQYAERHRLEILCHPSEYNKWKLTNSPTFLEVLEEFPSLRVSAGFLLSQLPILKPRYYSISSSRDCTPMEVHLTVAVLVYPTRDGQGPLHHGVCSTWLSNLKPQDPVPCFVRSAGNFKLPEDPSRPCILIGPGTGIAPFRSFWQQRLHDIKHKGLRGSRMTLVFGCRRPDEDHLYREEMLEMAQSGVLHEVHTAYSRLPGQPKVYVQDILRQQLASQVLRMLHEEQGHLYVCGDVRMARDVAHTLKHLVAAKLSLSEEQVEDYFFQLKSQKRYHEDIFGAVFPYEVKKDGAAKQPSDPRVPAAHGRS

SEQ ID NO: 61 - protein, 1156 aa, Bos taurus
MACPWQFLFKIKSQKVDLATELDINNNVGKFYQPPSSPVTQDDPKRHSPGKHGNESPQPLTGTVKTSPESLSKLDAPPSACPRHVRIKNWGSGVTFQDTLHQKAKGDLSCKSKSCLASIMNPKSLTIGPRDKPTPPDELLPQAIEFVNQYYGSFKEAKIEEHLARVEAVTKEIETTGTYQLTGDELIFATKQAWRNAPRCIGRIQWSNLQVFDARSCSTAQEMFEHICRHVRYATNNGNIRSAITVFPQRSDGKHDFRVWNAQLIRYAGYQMPDGSIRGDPANVEFTQLCIDLGWKPKYGRFDVLPLVLQADGRDPELFEIPPDLVLEVPMEHPRYEWFRELELKWYALPAVANMLLEVGGLEFPGCPFNGWYMGTEVGVRDFCDAQRYNILEEVGRRMGLETHKVASLWKDRAVVEINVAVLHSFQKQNVTIMDHHSAAESFMKYMQNEYRSRGGCPADWIWLVPPISGSITPVFHQEMLNYVLSPFYYYQVEPWKTHVWQDERRRPQRREIRFKVLVKAVFFASVLMHKAMASRVRATILFATETGRSETLAQDLGALFSCAFNPKVLCMDQYQLSHLEEEQLLLVVTSTFGNGDSPGNGEKLKKSLLMLKELTNTFRYAVFGLGSSMYPQFCAFAHDIDQKLSQLGASQLAPTGEGDELSGQEEAFRSWAVQTFKAACETFDVSGKHHIEIPKLYTSNVTWDPQHYRLVQDSEPLDLNKALSSMHAKHVFTMRLKSQQNLQSPKSSRTTLLVELSCEGSQAPSYLPGEHLGVFPCNQPALVQGILERVVDGPAPHQPVRLETLCENGSYWVKDKRLPPCSLSQALTYFLDITTPPTQLLLRKLAQLATEEAEKQRLETLCQPSDYNKWKFTNSPTFLEVLEEFPSLRVSASFLLSQLPILKPRYYSISSSRDLTPTEIHLTVAVLTYRTRDGQGPLHHGVCSTWLSSLKPQDPVPCFVRSASGFQLPEDRSRPCILIGPGTGIAPFRSFWQQRLHEAEHKGLQGGRMTLVFGCRRPEEDHLYWEEMLEMARKGVLHEVHTAYSRLPDQPKVYVQDILRQRLAGEVLRVLHEEQGHLYVCGDVRMARDVARTLKQLMATALSLNEEQVEDYFFQLKNQKRYHEDIFGAVFPYEVKKDGAAGLPSNPRAPGAHRS

SEQ ID NO: 62 - protein, 1136 aa, Gallus gallus
MLCPWQFAFKPHAVKNQSSEEKDINNNVEKDVKVHSFVKDDAKLHSLSKKQMKMSPIITSAEKHPQNGIKASNQISRCPRHVKVRNMENGSSLLDTLHLTAKEVINCRTRACQGALMTPKGLVRSTRDGPVPPAELLPQAVDFVKQYYSSFKELKIEEHLARLETVTKEIETTGTYHLTKDELIFAAKQAWRNAPRCIGRIQWSNLQVFDARDCKTAKEMFEYICRHIQYATNNGNIRSAITIFPQRTDGKHDFRVWNSQLIRYAGYQMPDGSVIGDPASVEFTKLCIELGWKPKYGRFDVVPLILQANGQDPEIFEYPPEIILEVPMEHPKYEWFKELDLKWYALPAVANMLLEVGGLEFTACPFNGWYMGTEIGVRDFCDVQRYNILKEVGRRMGLESNKLASLWKDRAVVEINVAVLHSFQKQNVTIMDHHSAAESFMKYMQNEYRVRGGCPADWVWIVPPMSGSITPVFHQEMLNYVLTPFFYYQVDAWKTHIWHDETRRPKKREIKLSILAKAVLLASLLLQKTMAARPKVTVIYATETGKSETLANSLCSLFSCAFNTKILCMDEYNISDLEKETLLLVVTSTFGNGDSPNNGKTLKNSLLTLKLLRKNIRYAVFGLGSTMYPEFCAFAHAIDQKLSQLGALQLTPVGEGDELNGQEEAFRTWAVTAFKTACDIFDIRGKNSIQLPEIYTSDDSWNPKKHRIVYDSQTMDLTKALSDIHGKNVIPMKLKFRQNLQSLKSSRVTILVKLSCETNQEVHYLPGEHIGISPGNQPELVHGLIARVKDAPPADQTIRLETCTEGGYWASEKKIPACTLSQALTYLLDITTPPTQQLLKKLSQLVTAEGDKQRLEVLCHSTEEYNKWKFYNRPNILEVLEEFPSAEVSTAFLLTQLPLLKPRYYSVSSSCDMTPREIHLTVAVVNYRTRDGQGPLHHGVCSTWLNKIALNETVPCFVRSADGFRLPKEPAKPCILIGPGTGIAPFRSFWQQRLYDLEKKGIKGGDMILLFGCRHPDMDHIYKEEVEEMKRKGVLKEVFTAYSRQPGQPKVYVQDILQNELETKVCNILHKEEGHLYVCGDVRMARDVAQTLKRMLVKKLNHTEQQAEEYFFQLKSQKRYHEDIFGAVFPHEVKRI

SEQ ID NO: 63 - protein, 1147 aa, Rattus norvegicus
MACPWKFLFRVKSYQGDLKEEKDINNNVEKTPGAIPSPTTQDDPKSHKHQNGFPQFLTGTAQNVPESLDKLHVTPSTRPQHVRIKNWGNGEIFHDTLHHKATSDISCKSKLCMGSIMNSKSLTRGPRDKPTPVEELLPQAIEFINQYYGSFKEAKIEEHLARLEAVTKEIETTGTYQLTLDELIFATKMAWRNAPRCIGRIQWSNLQVFDARSCSTASEMFQHICRHILYATNSGNIRSAITVFPQRSDGKHDFRIWNSQLIRYAGYQMPDGTIRGDPATLEFTQLCIDLGWKPRYGRFDVLPLVLQAHGQDPEVFEIPPDLVLEVTMEHPKYEWFQELGLKWYALPAVANMLLEVGGLEFPACPFNGWYMGTEIGVRDFCDTQRYNILEEVGRRMGLETHTLASLWKDRAVTEINAAVLHSFQKQNVTIMDHHTASESFMKHMQNEYRARGGCPADWIWLVPPVSGSITPVFHQEMLNYVLSPFYYYQIEPWKTHIWQDEKLRPRRREIRFTVLVKAVFFASVLMRKVMASRVRATVLFATETGKSEALARDLAALFSYAFNTKVVCMEQYKANTLEEEQLLLVVTSTFGNGDCPSNGQTLKKSLFMMKELGHTFRYAVFGLGSSMYPQFCAFAHDIDQKLSHLGASQLAPTGEGDELSGQEDAFRSWAVQTFRAACETFDVRSKHCIQIPKRYTSNATWEPEQYKLTQSPESLDLNKALSSIHAKNVFTMRLKSLQNLQSEKSSRTTLLVQLTFEGSRGPSYLPGEHLGIFPGNQTALVQGILERVVDCSSPDQTVCLEVLDESGSYWVKDKRLPPCSLRQALTYFLDITTPPTQLQLHKLARFATEETHRQRLEALCQPSEYNDWKFSNNPTFLEVLEEFPSLRVPAAFLLSQLPILKPRYYSISSSQDHTPSEVHLTVAVVTYRTRDGQGPLHHGVCSTWINNLKPEDPVPCFVRSVSGFQLPEDPSQPCILIGPGTGIAPFRSFWQQRLHDSQHRGLKGGRMTLVFGCRHPEEDHLYQEEMQEMVRKGVLFQVHTGYSRLPGKPKVYVQDILQKELADEVFSVLHGEQGHLYVCGDVRMARDVATTLKKLVAAKLNLSEEQVEDYFFQLKSQKRYHEDIFGAVFSYGAKKGNTLEEPKGTRL

SEQ ID NO: 64 - protein, 196 aa, Homo sapiens
PEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPAGTQGEQLEVPERLLVVLDMEEGTLGYAIGGTYLGPAFRGLKGRTLYPAVSAVWGQCQVRIRYLGERR

SEQ ID NO: 65 - protein, 182 aa, Mus musculus
DYCKPTRLDLLLDMPPVSYDVQLLHSWNNNDRSLNVFVKEDDKLIFHRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNG

SEQ ID NO: 66 - protein, 200 aa, Bos taurus
DYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNG

SEQ ID NO: 67 - protein, 200 aa, Homo sapiens; identical to SEQ ID NO: 66

SEQ ID NO: 68 - protein, 200 aa, Rattus norvegicus
DYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYVNG

SEQ ID NO: 69 - protein, 200 aa, Canis familiaris
DYCKPTRLDLLLDMPPVSYDIQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNG

SEQ ID NO: 70 - protein, 200 aa, Danio rerio
DYTKPSRLDMLLDMPSASQDIQVQHSWNNDDRSLNIFVKEDNKLIFHRHPVAQSTDAIRGRVGYTRGLHVWEISWAMRQRGTHAVVGVATGEAPLHSVGYTALVGNNSESWGWDLGRNKLYHDGKNQPSRTYPAFLEPDETFIVPDSFLVVLDMDEGTLSFIVDGQYLGVAFRGLKGKKLYPVVSAVWGHCEIRIRYING

SEQ ID NO: 71 - protein, 200 aa, Drosophila melanogaster
DFVKPARIDILLDMPPASRDLQLKHSWNSEDRSLNIFVKEDDKLTFHRHPVAQSTDCIRGKVGLTKGLHIWEIYWPTRQRGTHAVVGVCTADAPLHSVGYQSLVGSTEQSWGWDLGRNKLYHDSKNCAGVTYPAILKNDEAFLVPDKFLVALDMDEGTLSFIVDQQYLGIAFRGLRGKKLYPIVSAVWGHCEITMRYIGG

SEQ ID NO: 72 - protein, 203 aa, Danio rerio
DFFKPARLDLLLDMPPASYEQQLRHAWNSDDRSLNIFIKDDDKLTFHRHPVAQSTDGIRGRVGYTRGLHVWRIHWPARQRGTHAVVGVATGDAPLHSVGYTSLVGSDSESWGWDLGRNKLYHNSKNRPASSAPTYPSFLEPEEAFVLPDTLMVVLDMDEGTLSFMVDGQYLGVAFRGLKGKKLYPIVSAVWGHCEITMRYING

SEQ ID NO: 73 - protein, 200 aa, Mus musculus
EPGRPARLDQLLDMPAAGLAVQLRHAWNPEDRSLNVFVKDDDRLTFHRHPVAQSTDGIRGKVGHARGLHAWQIHWPARQRGTHAVVGVATARAPLHSVGYTALVGSDSESWGWDLGRSRLYHDGKNRPGVAYPAFLGPDEAFALPDSLLVVLDMDEGTLSFIVDGQYLGVAFRGLKGKKLYPVVSAVWGHCEVTMRYING

SEQ ID NO: 74 - protein, 200 aa, Rattus norvegicus; identical to SEQ ID NO: 73

SEQ ID NO: 75 - protein, 200 aa, Canis familiaris
EPGRPARLDQLLDMPAAGLAVQLRHAWNPEDRSLNVFVKDDDRLTFHRHPVAQSTDGIRGKVGHARGLHAWQINWPARQRGTHAVVGVATARAPLHSVGYTALVGSDAESWGWDLGRSRLYHDGKNRPGVAYPAFLGPEEAFALPDSLLVVLDMDEGTLSFVVDGQYLGVAFRGLKGKKLYPVVSAVWGHCEVTMRYING

SEQ ID NO: 76 - protein, 200 aa, Bos taurus
EPGRPARLDQLLDMPAAGLAVQLRHAWNPEDRSLNVFVKDDDRLTFHRHPVAQSTDGIRGKVGHARGLHAWQIHWPARQRGTHAVVGVATARAPLHSVGYTALVGSDAESWGWDLGRSRLYHDGKNRPGVAYPAFLGPEEAFALPDSLLVVLDMDEGTLSFVVDGQYLGVAFRGLKGKKLYPVVSAVWGHCEVTMRYING

SEQ ID NO: 77 - protein, 200 aa, Homo sapiens
EPGRPARLDQLLDMPAAGLAVQLRHAWNPEDRSLNVFVKDDDRLTFHRHPVAQSTDGIRGKVGHARGLHAWQINWPARQRGTHAVVGVATARAPLHSVGYTALVGSDAESWGWDLGRSRLYHDGKNQPGVAYPAFLGPDEAFALPDSLLVVLDMDEGTLSFIVDGQYLGVAFRGLKGKKLYPVVSAVWGHCEVTMRYING

SEQ ID NO: 78 - protein, 198 aa, Mus musculus
DFSPPEGLEELLSAPPPDLVAQRHHGWNPKDCSENIDVKEGGLCFERRPVAQSTDGVRGKRGYSRGLHAWEISWPLEQRGTHAVVGVATALAPLQADHYAALLGSNSESWGWDIGRGKLYHQSKGLEAPQYPAGPQGEQLVVPERLLVVLDMEEGTLGYSIGGTYLGPAFRGLKGRTLYPSVSAVWGQCQVRIRYMGE

SEQ ID NO: 79 - protein, 198 aa, Rattus norvegicus
DLSPPEGLEELLSAPPPDLGAQRHHGWNPKDCSENIDVKEGGLCFERRPVAQSTDGVRGKRGYSRGLHAWEISWPLEQRGTHAVVGVATALAPLQADHYAALLGSNSESWGWDIGRGKLYHQSKGLEAPQYPAGPQGEQLVVPERLLVVLDMEEGTLGYSIGGTYLGPAFRGLKGRTLYPSVSAVWGQCQVRIRYLGE

SEQ ID NO: 80 - protein, 198 aa, Homo sapiens
DLSCPEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPAGTQGEQLEVPERLLVVLDMEEGTLGYAIGGTYLGPAFRGLKGRTLYPAVSAVWGQCQVRIRYLGE

SEQ ID NO: 81 - protein, 198 aa, Bos taurus
DRSRPEGLEELLSAPPPDLGAQRRHGWNPKDCSENIEVKEGGLCFERRPVAQSTDGARGKRGYSRGLHAWEISWPREQRGTHAVVGVATARAPLQADHYAALLGSNSESWGWDIGRGKLYHQSKGPGAPQYPPGPQGEPLEVPERLLVVLDMEEGTLGYAVGGTYLGPAFRGLKGRTLYPAVSAVWGQCQVRINYLGE

SEQ ID NO: 82 - protein, 198 aa, Canis familiaris
DLSCPEGLEELLSAPPPDLGAQQRHGWNPKDCSENIEVKEGGLCFERRPVAQSTDGARGKKGYSRGLHAWEICWPREQRGTHAVVGVATALAPLQADHYAALLGSNSESWGWDIGRGKLYHQSKGPGAPQYPARPHGEQLEVPERLLVVLDMEEGTLGYAIGGTYLGPAFRGLKGRTLYPAVSAVWGQCQVRIRYLGG

SEQ ID NO: 83 - protein, 42 aa, Homo sapiens
AEPHSLLHLSRLCVRHNLGDTRLGQVSALPLPPAMKRYLLYQ

SEQ ID NO: 84 - DNA, 21 bp, Artificial Sequence
  shRNA oligonucleotide
ccagatgcagagaataaacta